(19) United States
(12) Patent Application Publication
(10) Pub. No.: US 2010/0278896 A1
(43) Pub. Date: Nov. 4, 2010

(54) SOLID COMPOSITIONS

(75) Inventors: Peng T. Khaw, London (GB); Stephen Brocchini, London (GB)

Correspondence Address: STITES & HARBISON, PLLC, 400 W MARKET ST, SUITE 1800, LOUISVILLE, KY, 40202-3352, US

(73) Assignee: UCL BUSINESS PLC, London (GB)

(21) Appl. No.: 12/743,147

(22) PCT Filed: Nov. 17, 2008
(86) PCT No.: PCT/GB2008/003851

§ 371 Date: Jul. 19, 2010

(30) Foreign Application Priority Data
Nov. 15, 2007 (GB) 0722484.3

(51) Int. Cl. A61K 9/00; A61K 31/56; A61K 39/395; C07K 16/00; C07C 259/04; A61K 31/16; A61K 31/404; C07D 209/20; C07K 16/46; C07J 5/00; A61K 31/573; C07D 239/553; A61K 31/513; C07C 59/64; A61K 31/192; C07K 16/22; A61P 35/00; A61P 17/02

(52) U.S. Cl. 424/423; 514/169; 424/130.1; 552/502; 530/387.1; 562/621; 514/575; 514/171; 530/388.1; 514/419; 548/494; 530/387.3; 424/133.1; 424/141.1; 552/566; 552/574; 514/180; 544/313; 514/274; 562/466; 514/569; 530/389.2; 424/158.1

(57) ABSTRACT

A solid, implantable dosage form comprising a therapeutically active agent in solid form, optionally with one or more pharmaceutically acceptable excipients, wherein the one or more excipients, when present, do not lead to a significant delay or prolongation of the release of active agent, as compared to an equivalent dosage form containing no excipients when tested in vitro.

The present invention relates to solid pharmaceutical compositions and, in particular, the use of substantially water insoluble therapeutically active agents for local delivery for preventing or treating disease. The present invention more specifically relates to solid matrix metalloproteinase (MMP) inhibitor compositions and their use in preventing scarring. The present invention also relates to specific MMP inhibitor solid dosage forms.

Therapeutic agents that are substantially water insoluble are generally delivered to the human or animal body in a suitable solvent, such as DMSO, etc. However, by delivering the therapeutic agent in a solution, the agent is usually administered systemically. If such a solution is administered locally, it generally only remains at the site of administration for a short period of time (i.e., a few minutes to a few hours). It is desirable to deliver therapeutic agents locally so that only the relevant part of the body is exposed to the agent. It is also important that any therapeutically active agent delivered to the body has a suitable dissolution profile enabling a therapeutically effective concentration of the active agent to be achieved for a sufficiently prolonged period of time to allow treatment. Numerous multicomponent and complicated drug formulations have been developed in an effort to resolve these issues; however, such formulations can be expensive, physically and chemically sensitive and labile, and specific to the therapeutically active agent being delivered.

One preferred aspect of the present invention concerns preventing or treating tissue scarring. The processes involved in scarring can play a part in treatment failure in a variety of situations. Furthermore, scarring appears to play a part in treatment failure in virtually every blinding disease in the world today. A very good example of the importance of healing and scarring in the eye is what happens after glaucoma surgery to create a fistula to reduce the pressure in the eye. The final eye pressure determines the success of the operation and is dependent on the healing and scarring process. The wound healing process that occurs in the eye after trabeculectomy starts after the initial conjunctival incision. Plasma proteins and blood cells are released in the wound area and a fibrin clot is formed. Neutrophils and macrophages are recruited at the wound area and degrade the clot by expressing several enzymes and MMPs such as MMP-8 and -9 among them.

Activation and migration of fibroblasts to the wound site also takes place. The fibroblasts in normal unwounded tissues are quiescent undifferentiated mesenchymal cells known as fibrocytes. They exist in low numbers in the subconjunctival connective tissue-Tenon's capsule (Wong et al. 2002). After their activation, these fibroblasts produce large amounts of extracellular matrix (ECM) molecules such as collagens, glucosaminoglucans and elastin. They also produce MMPs that facilitate cleavage of the ECM.

Many research groups have investigated the role of MMPs in wound healing after glaucoma filtration surgery (GFS) (Kawashima et al. 1998). With the use of monoclonal antibodies they observed staining for MMP-1, MMP-2, TIMP-1 and TIMP-2 in the cytoplasm of fibroblasts isolated from human subconjunctival connective tissue. Moreover, comparison between normal and healing conjunctiva has shown that the MMP-1 and TIMP-1 were located only in the healing subconjunctival tissue. Neither molecule was found in normal subconjunctival tissue nor in the conjunctival epithelium. Based on these results, a possible role for MMPs in post-operative subconjunctival scarring has been proposed.

Since these early studies the expression of other MMP molecules has been detected in cultured human Tenon's fibroblasts (HTF) (Mietz et al. 2003). MMP-1, -2, -3, -9, -14 and TIMP-1 and -2 are expressed from in vitro cultured HTF. During the fibroblast migration over the fibronectin interface, traction forces are generated in the underlying substrate leading to wound contraction (Harris, Stopak, & Wild 1981). Gradually the fibrovascular granulation tissue is formed and a part of the fibroblast population differentiates in the wound site to myofibroblasts due to mechanical stress and growth factor stimulation (mainly TGF-β and PDGF). After continuous remodeling of the granulation tissue and apoptosis of myofibroblasts, dense collagenous subconjunctival scar tissue is formed. Extended subconjunctival fibrosis and the contraction of the tissue is the end result. This causes loss of function of the bleb with subsequent increase of intraocular pressure (IOP).

Solutions of antimetabolites such as mitomycin C (MMC) and 5-fluorouracil (5-FU) have been shown to be effective in reducing the scarring after trabeculectomy (Dahlmann et al. 2005; Skuta et al. 1992). Many studies have been published by the inventors that describe the increase of the functioning period of the outflow channel in the bleb. Results indicate that a single five minute application of a 5-FU or mitomycin C solution during surgery reduces the healing response and decreases scar formation. It is thought this is mainly due to suppression of fibroblast proliferation, prolonging the bleb survival (Doyle et al. 1993; Khaw et al. 1994; Khaw et al. 1992). Unfortunately, severe complications often occur after treatments with these metabolites. The bleb often leaks and there other side effects including hypotony, endophthalmitis and excessive ocular cell apoptosis that can cause irreversible vision loss. Despite this, MMC and 5-FU are still used. Hence safer and more effective agents are needed to reduce scarring and to control healing after GFS.

Since MMPs take part in several pathological conditions, it is important to identify selective inhibitors that can be used therapeutically to control MMP activity in defined ways. The use of the natural TIMP inhibitors has significant disadvantages such as their high molecular weight and their poor oral bioavailability, which prevent their clinical use (Glasspool & Twelves 2001b).

To overcome these difficulties, synthetic compounds to block MMP activities (MMP inhibitors) have been designed. Some of the most well-known MMP inhibitors are Batimastat (BB-94), Marimastat (BB-2516), Prinomastat (AG3340), Tanomastat (BAY12-9566) (Glasspool & Twelves 2001a) and Ilomastat (GM6001) (Galardy et al. 1994d). These are hydroxamic acid derivatives that bind reversibly to the zinc in the active site of MMPs. Most of the potent inhibitors designed to date are right-side binders, as left-side binding is much weaker possibly due to its natural ability to prevent the carboxylate product of substrate cleavage from becoming a potent inhibitor of the enzyme (Skiles, Gonnella, & Jeng 2001).

MMPs play a significant role in wound contraction (Daniels et al. 2003; Porter et al. 1998). In particular, inhibition of MMPs reduced wound contraction in in vitro experiments using collagen I lattices as the wound contraction model (Scott, Wood, & Karran 1998). Both in vitro and in vivo studies have been performed in order to test the effect of MMP inhibitors in contraction models. Daniels et al., 2003, tested the effect of three MMP inhibitors—Ilomastat, BB-94 and BMS-275291 (Cell Tech) in HTF populated collagen gels. Observations revealed inhibition of the contraction of the gels with the application of all the three MMP inhibitors in a dose-dependent manner and Ilomastat was observed to be the most effective.

The tested MMP inhibitors were also found to have a non toxic and reversible effect and zymography results indicated significant reduction of the proteolytic activity of the detected MMP bands after the application of the MMP inhibitors. It was also shown that Ilomastat inhibited collagen production from fibroblasts in a dose-dependent manner. This was an important finding, as excessive collagen production and deposition at the incision area is mainly responsible for the bleb failure (Cordeiro et al. 2000; Daniels et al. 1998).

Administration of 17 injections of dissolved Ilomastat in DMSO in an in vivo 30 days rabbit contraction model after trabeculectomy was found to prolong significantly the bleb survival in comparison to the DMSO only control group as well as to have a lowering IOP effect throughout the experiment (Wong, Mead, & Khaw 2003). Histological findings showed that reduction of scar tissue formation in the Ilomastat treatment group occurred with decreased cellularity compared to the control group. There was also decreased cell apoptosis (that is known from other studies to be associated to MMC), decreased myofibroblasts in the wound area (possibly because of an inhibitory effect of Ilomastat in fibroblast migration) and a large bleb area compared to control group.

The necessity of comparison of the antiscarring effects of Ilomastat with MMC led to the design of a new comparative in vivo study (Wong, Mead, & Khaw 2005). The Ilomastat treated group had similar prolonged bleb survival and IOP lowering results as the MMC treated group. Importantly, this study showed that the morphology of the subconjunctival tissue was normal in the Ilomastat group but hypocellular in the MMC group. It is worth mentioning that in none of the inventors' in vivo experiments Ilomastat damaged conjunctiva, as can happen in the case of MMC.

The clinical use of Ilomastat for post surgical wound management may have advantages over the currently used cytotoxic antimetabolites. Ilomastat displays specific MMP inhibition and blocks the activation of fibroblasts. No reports of toxicity have been published, so it is possible that Ilomastat will be better tolerated for post-operative GFS treatment than the antimetabolites. There are several other challenges however that have to be addressed to increase the benefit of post-trabeculectomy treatment in order to reduce scarring (Wong, Mead, & Khaw 2005).

The use of MMP inhibitors in preventing tissue contraction is described in International Patent Application WO 95/24921.

Currently, a single administration of MMC is used during trabeculectomy beneath the scleral flap. Multiple, repetitive injections of an antimetabolite is not a viable choice due to toxicity associated with the drug, and to the discomfort and risk of infection to the patient caused by multiple injections. Furthermore, maintaining a constant local concentration of active agent in the bleb, the capacity of which is approximately 200 μl, is not possible by bolus injection. The reason is that there is aqueous outflow of 2 μl/min from the anterior chamber to the bleb, which means that the concentration of the injected agent would be quickly reduced. It is also not possible to continuously infuse the agent.

There is a need to develop a continuous prolonged drug release system that can be placed in the subconjunctival space after trabeculectomy.

The inventors' initial work was on developing a delivery system with Ilomastat. Ilomastat is known to inhibit in vitro contraction in collagen I gels in a dose dependent manner in concentrations ranging from 10-100 nM (Daniels et al. 2003 and International Patent Application WO 95/24921). Increased efficacy has been observed during in vivo studies with the administration of multiple injections of Ilomastat at a concentration of 100 nM (Wong, Mead, & Khaw 2005; Wong, Mead, & Khaw 2003). While this preliminary work established the favourable pharmacological effects of Ilomastat, the therapeutic concentration could only be achieved with injections that had been prepared from aqueous-DMSO solutions. DMSO has not been approved for ocular human use.

There is a need for a method of localised delivery of a substantially water insoluble therapeutically active agent for treating or preventing a disease. There is also a particular need for an agent that has suitable anti-scarring activity, low toxicity when implanted in the human or animal body, the active agent has low toxicity both locally and systemically, and an optimal dissolution profile for providing long term anti-scarring activity.

The present invention overcomes at least some of the problems associated with the prior art methods.

In accordance with a first aspect of the invention, there is provided a solid, implantable dosage form comprising a therapeutically active agent in solid form, optionally with one or more pharmaceutically acceptable excipients, wherein the one or more excipients, when present, do not lead to a significant delay or prolongation of the release of active agent, as compared to an equivalent dosage form containing no excipients when tested in vitro.

The dosage form of the first aspect is based on the surprising finding that it is possible to implant relatively simple solid dosage forms at selected sites in vivo and these dosage forms provide a steady release of active agent, without the need for complex sustained release formulations in which the release profile is controlled primarily by the excipients. The comparison in dissolution rates between excipient-containing and excipient-free dosage forms may be conducted using any suitable dissolution apparatus providing a flow of media which mimics the flow of in vivo media following tissue implantation, such as the flow-though rig described herein. The dissolution should be conducted at around 37 deg C., and in media of pH around 7.4.

The dosage form is preferably suitable for the localised prevention or treatment of a disease. It is possible that the dosage form of the first aspect may be implanted for the systemic delivery of an active agent. However, it is preferred that the dosage form is prepared with an amount of an appropriate therapeutic agent which makes it suitable for release and/or efficacy only in the locality of the implantation site.

In a preferred embodiment, the dosage form is suitable for ocular, periocular or intraocular implantation. For example, the dosage form may be suitable for implantation in the subconjunctival space.

In preferred embodiments, the dosage form is sterilised. Such treatment enables the dosage form to be safely implanted in a wider range of sites in vivo. The term ‘sterilised’ as used herein covers both dosage forms prepared by sterile manufacture, and those prepared by non-sterile manufacture which are subjected to a post-manufacturing sterilisation process, such as by gamma irradiation.

When the dosage form contains one or more excipients, it is preferred that these are biodegradable and/or bioresorbable following in vivo implantation. This has the advantage that the dosage form can be implanted and left to dissolve and/or biodegrade, without the need for a subsequent step of removal of any components of the dosage form after complete or partial release of the active agent. It is also an important consideration, for many active agents, that the excipients, when present, are not highly soluble or dispersible at the site of implantation; this avoids dose dumping and/or increased dissolution due to the dispersal of the active. The invention exploits the ‘non-sink’ conditions of the tissue into which implantation is made (for many actives, especially matrix metalloproteinase inhibitors). Depending on the solubility of the active agent concerned, and the flow of aqueous biological media through the tissue into which implantation is to be made (both of which may readily be determined), non-sink conditions can generally be achieved. Because the tissue is non-sink, it does not matter, as far as drug release is concerned, if the dosage form has excipient or not. Without excipient, the dosage form is more simple because only the active needs to dissolve. There is no need for consideration of other components dissolving and/or causing problems in vivo (e.g. inflammation). Indeed, in many instances, the only reason to use an excipient is to ensure the dosage forms are compliant with manufacturing specifications; in general, excipient use is primarily for processing considerations in fabricating the dosage form. In the vast majority of active agents of usefulness according to the invention, excipient use is not required to aid dissolution or release characteristics.

In certain embodiments, the dosage form is prepared by compression. In particular instances, the dosage form is a tablet.

Surprisingly, it has been found that a number of active agents, hitherto known to be formulated in solid dosage forms in which the majority of the dosage form comprises a variety of excipients, can be formulated as implantable tablets with little or no excipient content. This allows the dosage forms to be efficiently prepared using existing tableting apparatus, and also provides advantageous results in terms of dissolution profile of the dosage forms so prepared.

In some embodiments, the dosage form has a volume of between 0.1 mm³ and 1.5 cm³, and/or has a maximum dimension of 5 mm or less, and/or has a weight of 10 mg or less. Such limits allow the dosage form to be implanted in a wider variety of sites in vivo.

In particular embodiments, the dosage form is substantially free of excipients. It is a surprising finding that a variety of active agents can be formed into solid unit dosage forms, such as compressed dosage forms (e.g. tablets), and yet still provide a steady release of active agent following implantation in vivo.

In preferred embodiments, the active agent is substantially water insoluble. Such insolubility enhances the sustained release of active agent in the dosage forms of the invention. The term ‘substantially water insoluble’, as used herein, is intended to mean sparingly water-soluble (i.e., requires at least 30 parts water to dissolve one part of the therapeutic agent or, in other words, around 35 mg/ml or less), preferably slightly soluble (i.e., requires at least 100 parts water to dissolve one part of the therapeutic agent or, in other words, around 10 mg/ml or less), more preferably very slightly water-soluble (i.e., requires at least 1000 parts water to dissolve one part of the therapeutic agent or, in other words, around 1 mg/ml or less), and most preferably practically water-insoluble (i.e., requires at least 10,000 parts water to dissolve one part of the therapeutic agent or, in other words, around 0.1 mg/ml or less). The solubility is measured at room temperature (about 20° C.) using water that has a physiologically acceptable pH (i.e., between about 5.0 and 8.0).

In particular embodiments, the active agent is a matrix metalloproteinase (MMP) inhibitor, which may be a hydroxamic acid derivative that binds reversibly to zinc in the active site of matrix metalloproteinases, and/or which may be a right side binder.

In general, the therapeutically active agent can be any suitable agent that is a solid at ambient temperature and which can be formulated into a solid unit dosage form. Such a limitation can readily be assessed by the skilled formulator. The therapeutically active agent may be a naturally occurring agent or a synthetic agent. In may instances, the active agent will be at least partially crystalline. Preferably the therapeutically active agent is a synthetic chemical compound. For MMP inhibitors (and other receptor antagonists or enzyme inhibitors), agents with low Ki values, i.e., high pKi values are generally preferred. For example, ilomastat has a Ki of 0.4 nM against collagenase.

An advantage of the present invention is that relatively low solubility compounds can be successfully delivered by means of the described dosage form. Traditionally, such compounds (which are frequently encountered), have had to be formulated using high drug contents and/or complex mixtures of excipients to improve solubility and/or provide sustained release. Equally, in traditional formulation approaches to solid active agents, solubility and tissue permebaility characteristics of the active are key considerations. In the implantable dosage forms of the present invention, and the related methods and uses, the need for permeation through a mucosal membrane (e.g. from the gut) is not required. This allows the invention to have a very wide applicability.

Preferred agents include MMP inhibitors and other anti-scarring agents, steroids, antibiotics, anticancer agents, antibody molecules and anti-inflammatory agents. Anti-scarring agents include MMP inhibitors, which are defined below, antimetabolites such as MMC and 5-FU, and TGF beta. Suitable steroids include corticosteroids, such as dexamethasone, hyrdocortisone, prednisolone, triamcinolone and methylprednisolone. Suitable antibiotics include any of the generally used antibiotics, including beta-lactam antibiotics, e.g., penicillins, macrolide antibiotics, e.g., erythromycin, and doxycycline. Suitable anti-cancer agents include SFU, paclitaxel and chlorambucil.

Any antibody molecule may be used. The term “antibody molecule” encompasses polyclonal antibodies, monoclonal antibodies or antigen binding fragments thereof, such as Fv, Fab, F(ab′)2 fragments and single chain Fv fragments. Preferably the antibody molecules are lyophilised antibody molecules. The target antigen of the antibody determines the therapeutic activity of the antibody. Numerous therapeutic antibodies are known to those skilled in the art.

Suitable anti-inflammatory agents include steroidal and non-steroidal anti-inflammatory agents. Preferably the anti-inflammatory agents are non-steroidal agents such as naproxen, ibuprofen, diclofenac and ketorolac.

The therapeutically active agent is preferably an agent that is for administration locally to the site of the disease. For example, when the agent is an anticancer agent, it would be desirable to deliver the agent to the site of a tumour. Alternatively, when the therapeutically active agent is an anti-scarring agent or an anti-inflammatory agent it is for implantation at the site of surgery, trauma or inflammation to prevent or treat inflammation or tissue scarring.

The therapeutically active agent is for treating or preventing a disease. The disease to be prevented depends on the therapeutically active agent. For example, when the agent is an anti-inflammatory, the agent is used to treat or prevent inflammation. Inflammation may be associated with a variety of diseases, including asthma, arthritis, localised infections, tissue damage caused by surgery or trauma, etc. When the agent is an anti-cancer agent, the agent is used to treat or prevent cancer. The anti-cancer agent is preferably used to treat tumours. When the agent is an antibiotic, the agent is preferably used to treat infections. When the agent is an anti-scarring agent it is used to prevent or reduce tissue scarring caused by infection, surgery, trauma, etc. As will be apparent to those skilled in the art, active agents can have more than one therapeutic use. For example, 5-FU is both an anti-scarring agent and an anti-cancer agent.

In preferred embodiments of the first aspect, the active agent is an MMP inhibitor selected from the group consisting of ilomastat batimastat, marimastat, prinomastat, tanomastat, Trocade (cipemastat), AG 3340, CGs227023A, BAY 12-9566, and BMS-275291, or any functional derivatives thereof.

Notwithstanding the above preference, the matrix metalloproteinase (MMP) inhibitor can be any MMP inhibitor that can be formulated into a solid unit dosage form. The MMP inhibitor may be a natural or a synthetic MMP inhibitor. Naturally-occurring MMP inhibitors include α2-macroglobulin, which is the major collagenase inhibitor found in human blood. Numerous synthetic MMP inhibitors have been developed and are described in the literature. For example, U.S. Pat. Nos. 5,183,900, 5,189,178 and 5,114,953 describe the synthesis of Ilomastat (N[2(R)-2-(hydroxamidocarbonylmethyl)-4-methylpentanoyl-Ltryptophan methylamide), also known as GM6001 or Galardin, and other MMP inhibitors. Other MMP inhibitors based on hydroxamic acid are disclosed in International Patent Applications WO 90/05716, WO 90/05719 and WO 92/13831. Further synthetic MMP inhibitors include those described in European patent applications EP-A-126,974 and EP-A-159,396 and in U.S. Pat. Nos. 4,599,361 and 4,743,587. Yet another inhibitor is BB-94, also known as Batimastat (British Bio-technology Ltd.), see for example, European patent application EP-A-276436. International Patent Application WO90/05719 also discloses MMP inhibitors 4-(N-hydroxyamino)-2R-isobutyl-3S-(thio-phenylthiomethyl)succinyl]-L-phenylalanine-N-methylamide and 4-(N-hydroxyamino)-2R-isobutyl-3S(thiomethyl)succinyl]-L-phenylalanine-N-methyl-amide. International Patent Application WO90/05716 discloses MMP inhibitors 4-(N-hydroxyamino)-2R-isobutylsuccinyl-Lphenylalanine-N-(3-aminomethylpyridine) amide and [4-Nhydroxyamino)-2R-isobutyl-3S-methylsuccinyl]-Lphenylalanine-N-4-(2-aminoethyl)-morpholino amide.

The properties of natural and synthetic collagen inhibitors may vary. Individual inhibitors often have different specificities and potencies. Some inhibitors are reversible, others are irreversible. In general the more potent an inhibitor's inhibitory effects the better. Generally a broad spectrum MMP inhibitor, for example, Ilomastat, is preferred.

The MMP inhibitor may be an anti-MMP polyclonal or monoclonal antibody molecule. Antibodies which are specific for a particular MMP may be made and the use of such specific inhibitors may be preferred under certain circumstances. For example, an antibody to MMP1, MMP2 or MMP3 (collagenase, 72 kD gelatinase or stromelysin respectively) or a mixture of two or more thereof may be used. Methods for generating such anti-MMP antibodies are well known to those skilled in the art.

Preferably the MMP inhibitor is any one of the synthetic inhibitors mentioned above. Preferred inhibitors include peptide hydroxamic acids or pharmaceutically acceptable derivatives thereof. Especially preferred are those compounds that are described and claimed in U.S. Pat. Nos. 5,189,178; 5,183,900 and 5,114,953. Those with low Ki values, i.e., high pKi values are also generally preferred. Preferably, the MMP inhibitor is a hydroxamic acid derivative that binds reversibly to zinc in the active site of the MMPs, and more preferably a right side binder.

As mentioned above, in a particularly preferred embodiment, the MMP inhibitor is selected from the group consisting of Batimastat, Marimastat, Prinomastat, Tanomastat, Trocade, AG 3340, CGs227023A, BAY 12-9566, BMS-275291, and Ilomastat, or any functional derivates thereof. More preferably, the MMP inhibitor is Ilomastat, or any functional derivatives thereof. Functional derivatives of the various MMP inhibitors are well known to those skilled in the art. For example, functional derivatives of Ilomastat are disclosed in U.S. Pat. No. 5,183,900. Ilomastat is especially preferred because it is one of the most potent collagenase inhibitors known at present. However, for certain applications it may be preferable to use a less potent (weaker) inhibitor.

Studies by the inventors have demonstrated that Ilomastat can inhibit MMPs during subconjunctival wound healing without toxic effect. For these reasons the inventors initially focused on Ilomastat for use for scarring inhibition.

As indicated above, Ilomastat (molecular formula C₂₀H₂₈N₄O₄, 388.47 g/mol) is a peptide analogue with the formal chemical name of N-[(2R)-2-(hydroxamidocarbonylmethyl)-4-methylpentanoyl]-L tryptophan methylamide. It is a broad spectrum hydroxamate MMP inhibitor (Galardy et al. 1994a). The reported Ki values are as follows: Human MMP-1 (Fibroblast collagenase): 0.4 nM, Human MMP-3 (Stromelysin): 27 nM, Human MMP-2 (72 kDa gelatinase): 0.5 nM, Human MMP-8 (Neutrophil collagenase): 0.1 nM, Human MMP-9 (92 kDa gelatinase): 0.2 nM (Galardy et al. 1994c).

The solid dosage form of the present invention may comprise more than one therapeutically active agent, e.g., more than one MMP inhibitor or two or more different classes of therapeutically active agents. However, it is preferred that the solid form only comprises one therapeutically active agent, e.g., an MMP inhibitor.

In accordance with a second aspect of the invention, there is provided a dosage form according to the first aspect, for use in therapy.

In particular, when the dosage form contains an MMP inhibitor, it is preferably for use in preventing or reducing tissue scarring. In certain embodiments, the scarring is ocular, periocular or intraocular. In particular embodiments, the dosage form is implanted following glaucoma filtration surgery. The dosage form may, in instances such as those described, be implanted in the subconjunctival space.

It has been found that by providing the MMP inhibitor in a solid dosage form that a slow dissolution rate is achieved enabling the required in situ concentration of the MMP inhibitor to be achieved for at least 30 days. Such a slow dissolution rate results in the prevention or a substantial reduction of scarring leading to a better outcome for the patient being treated. With the previous methods of injecting solutions of the MMP inhibitor, clearance of the MMP inhibitor occurs within minutes. Even when slow release gels are used to provide the MMP inhibitor, clearance occurs within about 3-6 hours. By providing the MMP inhibitor in a solid dosage form, clearance does not occur for over 30 days. A further advantage of appropriate dosage forms of the invention is that the solid dosage form does not need to be removed as it completely dissolves and/or biodegrades in situ.

The present invention avoids the inconvenient and dangerous practice of giving multiple injections of an anti-scarring agent to the eye. Furthermore, by reducing the individual's exposure to the anti-scarring agent the risk of systemic complications (such as arthritis) are avoided.

The invention also provides the use of a dosage form according to the first aspect, in the preparation of a medicament for implantation for the localised prevention or treatment of a disease. In particular embodiments, particularly when the active agent is an MMP inhibitor, the medicament may be for implantation for the localised treatment or prevention of scarring in the tissue.

In a related manner, the invention also provides a method of locally preventing or treating a disease in a patient in need thereof, the method comprising administering a solid dosage form according to the first aspect to said patient, by implantation, in an amount sufficient to prevent or treat the disease. In preferred embodiments, the active agent is an MMP inhibitor, and the dosage form is administered for locally treating or preventing scarring in said patient. In such an instance, the dosage form may be administered by ocular, periocular or intraocular implantation, for example, by being implanted in the subconjunctival space. The scarring to be prevented or treated may be that following glaucoma filtration surgery.

In a third aspect, the present invention also provides the use of an MMP inhibitor in the manufacture of a solid, implantable medicament for preventing or reducing tissue scarring, by local implantation. Similarly, the invention provides an MMP inhibitor, for use in the prevention or reduction of tissue scarring, wherein the MMP inhibitor is formulated as a solid, implantable medicament, optionally containing one or more pharmaceutically acceptable excipients, for local implantation. A method of locally preventing or treating tissue scarring in a patient in need thereof, the method comprising administering a solid, implantable dosage form comprising a matrix metalloproteinase inhibitor, optionally with one or more pharmaceutically acceptable excipients, by local implantation.

In accordance with a fourth aspect of the invention, there is provided a solid, implantable dosage form comprising a therapeutically active agent in solid form, optionally with one or more pharmaceutically acceptable excipients, for use in therapy by ocular, periocular or intraocular implantation. Similarly, the invention provides the use of a solid, implantable dosage form comprising a therapeutically active agent in solid form, optionally with one or more pharmaceutically acceptable excipients, for the preparation of a medicament for the localised prevention or treatment of a disease by ocular, periocular or intraocular implantation. Also provided is a method of locally preventing or treating a disease in a patient in need thereof, the method comprising administering a solid, implantable dosage form comprising a therapeutically active agent in solid form, optionally with one or more pharmaceutically acceptable excipients, by ocular, periocular or intraocular implantation.

The fourth aspect is based on the surprising finding that a solid unit dosage form, containing an active agent in solid form, may be implanted at an appropriate ocular, intraocular or periocular site for the release of the active agent in the locality thereof. The active agent is preferably substantially water insoluble (as defined above). Such a characteristic provides for a longer and more steady release of active agent from the dosage form. In preferred embodiments of the third aspect, the active agent is a matrix metalloproteinase inhibitor. The MMP inhibitor may be as defined above in relation to the first aspect.

In a fifth aspect, the present invention provides a solid, implantable dosage form comprising a matrix metalloproteinase inhibitor, optionally with one or more pharmaceutically acceptable excipients, which is sterilised. The sterilisation of such a dosage form allows it to be implanted in sterile sites in vivo.

The invention also provides the use of a matrix metalloproteinase inhibitor in the manufacture of a solid dosage form as described above.

Also provided is a method of manufacturing a dosage form according to the fifth aspect, the method comprising:
i. forming a compressed dosage form, such as a tablet, containing the matrix metalloproteinase inhibitor and the excipients, when present, and
ii. sterilising the compressed dosage form by irradiating it with gamma radiation.

Furthermore, the invention provides a kit comprising a dosage form as described above and containing an MMP inhibitor, together with surgical equipment necessary for performing glaucoma filtration surgery.

The present invention also provides a method of preventing or reducing tissue scarring in a patient in need thereof comprising administering a matrix metalloproteinase inhibitor in a solid dosage form to said patient in an amount sufficient to prevent or reduce tissue scarring.

The solid dosage form of the present invention can, unless otherwise specified, be any solid dosage form, such as a tablet, that has the desired dissolution rate. The desired dissolution rate is one that allows a therapeutically effective concentration of the therapeutic agent to be released into the surrounding media for a substantial period of time. For example, at least one hour, more preferably at least one day, even more preferably for at least 5 days, more preferably at least 20 days, more preferably at least 30 days and, in some instances, up to 60 days. A variable dosing regimen may also be employed. For example, it may be possible, e.g. following surgery on a site, to implant a series of, say, 5 tablets, each of which provides 5 day release. These tablets may contain various doses. This will enable around 25 days of ongoing treatment using the active agent (e.g. MMP inhibitor), potentially using different concentrations thereof.

It has been found that when the therapeutically active agent is Ilomastat, an MMP inhibitory concentration of 10 μM is maintained for at least 30 days using a solid dosage form having a weight of about 2 to 5 mg. The concentration of the active agent that is maintained in situ will vary depending on the solubility of the agent and on the particular flow rate of fluid within the tissue wherein the solid dosage form is implanted.

Preferably the solid dosage form is suitable for implantation into a tissue, wherein on implantation it is slowly dissolved. Preferably the solid dosage form dissolves over a period of at least one day, preferably at least 5 days, more preferably at least 10 days, more preferably at least 20 days and most preferably at least 30 days and, in some instances, up to 60 days.

The shape of the solid dosage form can affect the dissolution rate by changing the surface area of the solid dosage form. The solid dosage form may be coated with a polymer that affects the dissolution rate. Such polymers are well known to those skilled in the art. Preferably, however, the solid dosage form is not coated with a polymer. The use of such polymers is generally not preferred as on clearance from the tissue a local inflammatory response may be induced, especially in the case of degradable polymers where degradation products could display toxicity. Another advantage with using an excipient and/or coating free tablet is that a proteinacious capsule does not form around the dosage form in vivo. Most implantables cause a foreign body response leading to capsule formation, and it is anticipated that most coatings will result in capsule formation when left in tissue—this being a form of inflammatory response.

The concentration of the therapeutically active agent to be delivered in order to prevent or treat the disease can be determined using standard techniques; however, when the active agent is an MMP inhibitor, generally, the concentration required to prevent or reduce tissue scarring is about 1 μM to about 1000 μM, more preferably about 10 μM to about 500 μM.

The shape of the solid dosage form will vary depending on the intended use. For example, if the solid dosage form is to be used to prevent tissue scarring after GFS, it is preferably of a shape and size enabling it to be delivered to the subconjunctival space. For example, it is preferred that the solid dosage form is a tablet having a diameter of 5 mm or less and a thickness of 2 mm or less. Preferably the tablet has a diameter of between 0.1 and 4mm with a thickness of between 0.1 and 1 mm. The shape of the solid dosage form will vary depending on the disease to be prevented or treated. The solid dosage form may be sized to enable it to be injected into the tissue to be treated, e.g., a tumour tissue, the vitreous humor, etc.

The present invention provides a substantially water insoluble therapeutically active agent in a solid dosage form for localised prevention or treatment of a disease.

It has been found that by providing a substantially water insoluble therapeutically active agent in a solid dosage form that a slow dissolution rate is achieved enabling the required in situ concentration of the agent to be achieved for a therapeutically effective time. The slow dissolution rate results in a prolonged exposure of the localised area of the body to the agent resulting in more effective localised treatment. A further advantage is that the solid dosage form does not need to be removed as it dissolves in situ. The present invention avoids the inconvenient practice of giving multiple injections of a therapeutically effective agent to an individual patient. Furthermore, by reducing the individual's exposure to the agent the risk of systemic complications are avoided.

The present invention also provides the use of a substantially water insoluble therapeutically active agent in the manufacture of a solid medicament for local delivery for preventing or treating a disease.

The present invention also provides a method of preventing or treating a disease in a patient in need thereof comprising locally administering a substantially water insoluble therapeutically active agent in a solid dosage form to said patient in an amount sufficient to prevent or treat the disease. The term “substantially water insoluble” is defined above.

The solid dosage form of the invention preferably has an overall volume of between 0.1 mm³ and 1.5 cm³, more preferably between 0.5 mm³ and 1 cm³. The solid dosage form may comprise one or more excipients but preferably is substantially excipient free. The term “substantially excipient free” means that the solid dosage form comprises less than 50% (w/w) excipients, preferably less than 40% (w/w) excipients, more preferably less than 10% (w/w) excipients, and most preferably the solid dosage form comprises at most trace amounts (1-2% (w/w)) of excipients. As described above, dosage forms of the invention may contain excipients, if necessary in levels above these limits, provided that the excipients are preferably bioresorbable and/or biodegradable in vivo. It has surprisingly been found that a solid dosage form consisting entirely of an MMP inhibitor, has the correct dissolution rate for preventing or reducing tissue scarring.

Suitable excipients are well known to those skilled in the art and include any conventional non-toxic pharmaceutically-acceptable carriers, adjuvants or vehicles. For example, pharmaceutically acceptable carriers, adjuvants and vehicles that may be used, include, but are not limited to, ion exchangers, alumina, aluminum stearate, lecithin, buffer substances such as phosphates, glycine, sorbic acid, potassium sorbate, partial glyceride mixtures of saturated vegetable fatty acids, sodium chloride, zinc salts, colloidal silica, magnesium trisilicate, polyvinyl pyrrolidone, cellulose-based substances, ethylcellulose, medium or high molecular weight (e.g. number average molecular weight of 600 or higher), polyethylene glycol, sodium carboxymethylcellulose, polyacrylates, waxes, solid polyoxylethylene-polyoxypropylene-block copolymers, wool fat, lactose and corn starch. Preferred excipients are biodegradable and/or bioresorbable from the implantation site in vivo.

The solid dosage form may comprise one or more additional active agents. Suitable additional active agents include antimetabolites, cytotoxic agents, anti-growth factors (e.g., TGFbeta, VEGF, etc.) or any other agents that may assist in the therapeutic treatment. For example, when the therapeutic agent is a MMP inhibitor, it is preferred that the additional active agent also prevents tissue scarring. However, it is preferred that the only active agent contained within the solid dosage form is the substantially water insoluble therapeutic agent, e.g., a MMP inhibitor.

The weight of the solid dosage form will vary depending on its intended use and on the amount of excipients or additional active agents that may be present. For example, if the solid dosage form is to be used to prevent tissue scarring during GFS, and consists entirely of the substantially water insoluble therapeutic agent, e.g., a MMP inhibitor, it is preferred that the solid dosage form weighs less than 10 mg, more preferably less than 6 mg, most preferably between 1 and 5 mg. The weight of the solid dosage form will vary depending on its intended use. Preferably the solid dosage form comprises between 1 and 5 mg of the substantially water insoluble therapeutic agent, e.g., MMP inhibitor. As the solid dosage form is, in use, positioned at a site within the body where the disease has occurred, or is likely to occur, the solid dosage form is preferably sterilized. The solid dosage form can be sterilized using any standard technique. Preferably, the solid dosage form is sterilized using gamma radiation.

According to the preferred embodiment of the present invention, the substantially water insoluble therapeutic agent is an MMP inhibitor for preventing or reducing tissue scarring. Any type of tissue scarring can be prevented or reduced using the solid dosage form of the MMP inhibitor described herein.

Scarring frequently occurs in the healing of burns. The burns may be chemical, thermal or radiation burns and may be of the eye, the surface of the skin or the skin and the underlying tissues. It may also be the case that there are burns on internal tissues, for example, caused by radiation treatment. Scarring may lead to physical and/or cosmetic problems, for example, loss of movement and/or disfigurement.

Scarring also occurs when producing skin grafts. Skin grafts may be applied for a variety of reasons and scarring may lead to both physical and cosmetic problems. It is a particularly serious problem where many skin grafts are needed as, for example, in a serious burns case.

Specific types of tissue scarring that can be prevented or reduced include ocular tissue scarring following eye surgery. Most forms of eye surgery cause some tissue scarring. For example, glaucoma filtration surgery (GFS) to create new drainage channels often fails due to scarring of tissues. A method of preventing scar tissue forming is therefore invaluable. Scar tissue may also be formed after corneal trauma or corneal surgery, for example laser or surgical treatment for myopia or refractive error. Opacification and cataract extraction can also cause scarring. Scar tissue may also be formed on/in the vitreous humor or the retina, for example, that which eventually causes blindness in some diabetics and that which is formed after detachment surgery, called proliferative vitreoretinopathy. Other types of scarring that may be prevented or reduced include scarring formed in the orbit or on eye and eyelid muscles after squint, orbital or eyelid surgery, or scarring of the conjunctiva which occurs in thyroid eye disease as may happen after glaucoma surgery or in cicatricial disease, inflammatory disease (e.g., pemphigoid), or infective disease (e.g., trachoma). In addition, the preparation of local ocular environments so as to make them permissive for tissue regeneration could benefit from the dosage forms of the invention.

Scarring is also associated with retinopathy of prematurity, macula degeneration, and myopia. Scarring of the optic nerve can also occur in glaucoma.

Another form of scarring is cicatricial contraction, namely contraction due to shrinkage of the fibrous tissue of a scar. In some cases the scar may become a vicious cicatrix, a scar in which the contraction causes serious deformity. A patient's stomach may be effectively separated into two separate chambers in an hour-glass contracture by the contraction of scar tissue formed when a stomach ulcer heals. Obstruction of passages and ducts, cicatricial stenosis, may occur due to the contraction of scar tissue. Contraction of blood vessels may be due to primary obstruction or surgical trauma, for example, after surgery or angioplasty. Stenosis of other hollow visci, for examples, ureters, may also occur. Problems may occur where any form of scarring takes place, whether resulting from accidental wounds or from surgery.

Solid dosage forms of the MMP inhibitors, may be used wherever scar tissue is likely to be formed, is being formed, or has been formed.

Scarring is also involved in conditions of the skin and tendons which involve contraction of collagen-comprising tissues, include posttrauma conditions resulting from surgery or accidents, for example, hand or foot tendon injuries, post-graft conditions and pathological conditions, such as scleroderma, Dupuytren's contracture and epidermolysis bullosa.

The solid dosage form of the MMP inhibitor is preferably used to treat or prevent tissue scarring associated with a chemical burn, a thermal burn or a radiation burn, a skin graft, a post-trauma condition resulting from surgery or an accident, glaucoma surgery, diabetes associated eye disease, scleroderma, Dupytren's contracture, epidermolysis bullosa or a hand or foot tendon injury. Preferably treatment should take place as early as possible, advantageously as soon as, and most advantageously before, the first signs of scarring. As indicated above, the solid dosage form is preferably for implantation at the site of surgery to prevent or reduce tissue scarring.

It is particularly preferred that the solid dosage form comprising the MMP inhibitor is for ocular delivery and for preventing scarring of eye tissue. Accordingly, the solid dosage form comprising the MMP inhibitor is preferably used to prevent or reduce ocular tissue scarring following eye surgery, especially following GFS. In particular, it has been found that by placing the solid dosage form within the subconjuctival space following GFS causes the slow release of the MMP inhibitor into the aqueous humor. The presence of the MMP inhibitor prevents the bleb (tissue covering the surgical incision) from scarring and thereby prevents fluid from passing out of the aqueous humor through the incision.

In a preferred embodiment of the present invention, the solid dosage form of the substantially water insoluble therapeutic agent, e.g., MMP inhibitor, consists essentially of the substantially water insoluble therapeutic agent, e.g., MMP inhibitor. The term “consists essentially of” as used herein means that the solid dosage form consists of the substantially water insoluble therapeutic agent, e.g., MMP inhibitor with only trace amounts (up to about 1 to 2% (w/w)) of other components.

The present invention also provides a solid pharmaceutical composition comprising a substantially water insoluble therapeutic agent which is in the form of an implantable tablet. Preferably the tablet is 5 mm or less in diameter and preferably also has a thickness of 2 mm or less. The tablet preferably has an overall volume of between 0.1 mm³ and 1.5 cm³. The therapeutic agent is as defined above. As indicated above, the tablet may comprise excipients and other active agents; however, preferably the tablet is substantially excipient free and consists essentially of the therapeutically active agent.

In a preferred embodiment, the present invention also provides a solid, implantable pharmaceutical composition comprising a matrix metalloproteinase inhibitor which is in the form of a tablet. Preferably the tablet is 5 mm or less in diameter and preferably also has a thickness of 2 mm or less. The tablet preferably has an overall volume of between 0.1 mm³ and 1.5 cm³.

The MMP inhibitor is as defined above. The tablet is preferably sized to enable it to be inserted into the subconjunctival space in order to prevent tissue scarring following eye surgery, especially GFS. As indicated above, the tablet may comprise excipients and other active agents; however, preferably the tablet is substantially excipient free and consists essentially of the MMP inhibitor.

The present invention also provides a solid pharmaceutical composition comprising a substantially water insoluble therapeutic agent which is in the form of a tablet that weighs less than 10 mg, preferably less than 6 mg.

The therapeutic agent is as defined above. As indicated above, the tablet may comprise excipients and other active agents; however, preferably the tablet is substantially excipient free and consists essentially of the therapeutic agent.

In a preferred embodiment, the present invention also provides a solid pharmaceutical composition comprising a matrix metalloproteinase inhibitor which is in the form of a tablet that weighs less than 10 mg, preferably less than 6 mg.

The MMP inhibitor is as defined above. As indicated above, the tablet may comprise excipients and other active agents; however, preferably the tablet is substantially excipient free and consists essentially of the MMP inhibitor.

The present invention also provides a sterilized solid pharmaceutical composition comprising a substantially water insoluble therapeutic agent. Preferably the substantially water insoluble therapeutic agent is a matrix metalloproteinase inhibitor. The MMP inhibitor is as defined above. Preferably the pharmaceutical composition is in the form of a tablet. As indicated above, the pharmaceutical composition may comprise excipients and other active agents; however, preferably the pharmaceutical composition is substantially excipient free and consists essentially of the substantially water insoluble therapeutic agent as the sole active agent. It is preferred that the solid pharmaceutical composition is sterilized by exposure to gamma radiation.

The present invention also provides a method of manufacturing a sterilized solid pharmaceutical composition comprising a substantially water insoluble therapeutic agent comprising:
i. forming a solid tablet of the substantially water insoluble therapeutic agent; and
ii. irradiating the tablet with gamma radiation to sterilize the tablet.

The method of the present invention enables the manufacture of a sterilized solid pharmaceutical composition for preventing or reducing tissue scarring. The step of forming the solid tablet of the substantially water insoluble therapeutic agent can be performed using any suitable technique. Preferably, the solid tablet is formed by compressing the substantially water insoluble therapeutic agent into a solid tablet using a punch-die or other suitable technique. The step of irradiating the tablet with gamma radiation preferably comprises subjecting the tablet to a 25 KGy dose to ensure sterilization, although lower doses may be sufficient. The therapeutic agent is as defined above, and is preferably a MMP inhibitor. As indicated above, the tablet may comprise excipients and other active agents; however, preferably the tablet is substantially excipient free and consists essentially of the substantially water insoluble therapeutic agent.

The present invention also provides a kit comprising a solid dosage form comprising a MMP inhibitor and surgical equipment necessary for performing glaucoma filtration surgery.

The MMP inhibitor is as defined above. It is also preferred that the solid dosage form is as defined above. The kit may comprise a plurality of the solid dosage forms, wherein a number of the solid dosage form may be implanted in the patient depending on the dosage required. The kit may also comprise instructions indicating how to use the solid dosage form.

Due to the small volume and the low aqueous flow characteristics of numerous body tissues, e.g., the subconjunctiva, non-sink conditions will exist. The rate determining step for the dissolution of the solid form of most active agents will be caused by these non-sink conditions. Dissolution in conditions where flow characteristics are thought to be within a consistent range will be primarily linear. This will prevent dose dumping and burst release kinetics and allow for a constant, sustained concentration of the active agent. Surprisingly there is no local contact tissue toxicity observed when using a tablet dosage form that is devoid of excipients. Also surprising is that small tablets can be fabricated that do not crumble or fall apart. Without being bound to any particular theory for this, it is presumed that this is due to trace residual water and the poorly soluble characteristics of the biologically active substance. The lack of excipients avoids the need to ensure the active is miscible and compatible with its excipients. This is typically required to ensure that phase separation of the active does not occur in the final dosage form.

Using a substantially water insoluble therapeutic agent, such as an MMP inhibitor, without excipients is surprising because it is stable in this form and maintains its activity. This is surprising because generally it would be expected that excipients would be needed to maintain a stable dispersion of the active and to prevent aggregation phenomena. So it is surprising that in a solid form designed for implantation that is predominantly devoid of excipients, that efficacy is observed without the need for repeat administrations of the active substance.

Since the dosage form is designed for use in the non-sink conditions inherent in the subconjunctiva, and in tissue generally, then use of a solid tablet form that is fabricated predominantly from the active substance will be optimal for maintaining a prolonged and consistent local concentration of the biologically active substance.

In accordance with a sixth aspect of the present invention, there is provided a pharmaceutical composition in solid unit dose form comprising an antibody, in solid form, optionally together with one or more pharmaceutically acceptable excipients.

The term ‘antibody’, which is synonymous with ‘antibody molecule’, has the same meaning as used in relation to the first aspect of the invention.

Hitherto, therapeutic or diagnostic antibodies have generally been formulated and administered as aqueous solutions. In certain cases, the antibody is presented as a freeze dried solid, but this solid must be reconstituted before use and a suitable dose extracted from the solution resulting therefrom. The inventors have surprisingly found that it is possible to formulate an antibody as a solid unit dosage form, with retention of antigen binding, and with suitable release characteristics for in vivo use. Furthermore, by formulating the antibody as a solid unit dose, it is possible to achieve a sustained release of the antibody following implantation in vivo; such release is not achievable with an aqueous injectable formulation. Such results are also achievable with other protein-based therapeutic or diagnostic agents.

In certain embodiments, the antibody is a monoclonal antibody. In particular, the antibody may be indicated for the treatment or prevention of a neoplastic disease, and may, for example, be an anti-VEGF antibody. An example of an anti-VEGF antibody is bevacizumab (Avastin).

The composition of this aspect of the invention is preferably sterilised.

When one or more excipients are present, these are preferably biodegradable and/or bioresorbable following in vivo implantation. In certain embodiments, the composition is substantially free of excipients (as defined above). In some embodiments, certain excipients, such as stabilising saccharides (e.g. trehalose), buffer salts, surfactants and/or similar, relatively soluble excipients which would typically be included in an aqueous injectable formulation of antibody, may be present, in some cases in significant amounts, without significantly affecting the advantageous properties of the composition of the invention. Indeed, in some instances, the incorporation of excipients can be used to improve and/or control the release of antibody from the composition. Thus, it has been found that hydrophilic polymers, such as hyaluronic acid, can be included in antibody tablet compositions of the invention, and can lead to an enhancement of antibody release when present in an appropriate amount. In greater amounts, hydrophilic polymers such as hyaluronic acid may be capable of producing a more sustained release of the antibody.

The composition of this aspect may be prepared by compression. A preferred composition of this type is a tablet. In any event, each solid unit dosage form preferably has a volume of between 0.1 mm³ and 1.5 cm³, and/or has a maximum dimension of 5 mm or less, and/or has a weight of 10 mg or less.

The composition of this aspect may contain one or more additional therapeutically active ingredients, which may or may not be an antibody, and which may or may not be in solid form.

The invention also provides a composition according to the sixth aspect, for use in therapy. In addition, the invention provides a composition according to the sixth aspect, for use in the treatment or prevention of a neoplastic disease. Similarly, the invention provides a method of treating or preventing a neoplastic disease in a patient in need thereof, the method comprising administering to said patient a pharmaceutical composition according to the sixth aspect.

In accordance with a seventh aspect of the invention, there is provided a solid, implantable, dosage form comprising a therapeutically active agent in solid form, optionally with one or more pharmaceutically acceptable excipients, wherein the one or more excipients, when present, do not control the release of the active agent by means of the chemical or biochemical degradation of one or more of the excipients. The dosage form is preferably sterilised.

In accordance with an eighth aspect of the invention, there is provided a solid, implantable, dosage form comprising a therapeutically active agent in solid form, optionally with one or more pharmaceutically acceptable excipients, wherein the dosage form is prepared by compression. The dosage form is preferably sterilised.

In accordance with a ninth aspect of the present invention, there is provided a pharmaceutical composition in solid unit dose form comprising a protein therapeutic or diagnostic agent, such as an antibody, in solid form, optionally together with one or more pharmaceutically acceptable excipients, wherein the dosage form is prepared by compression. The dosage form of this aspect is preferably in the form of a tablet. The dosage form of this aspect is preferably substantially excipient-free. The dosage form is also preferably sterilised. The dosage form is preferably implantable, and preferably has one or more of the additional features described above regarding suitability for implantation.

In accordance with a tenth aspect, the invention also provides a method of delivering a therapeutically active agent to an in vivo site for local prevention or treatment of a condition affecting that site, the method comprising implanting at the site a solid dosage form comprising the therapeutically active agent in solid form, optionally together with one or more pharmaceutically acceptable excipients. In certain embodiments, the dosage form is substantially excipient free. In certain embodiments, the excipients are non-polymeric.

The present invention is now described by way of example only with reference to the following Figures.

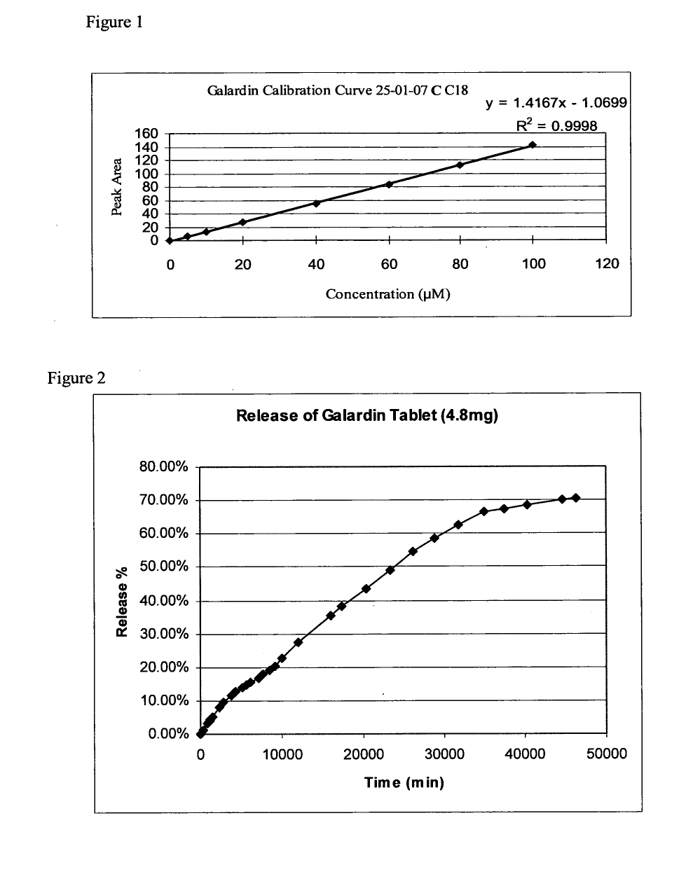

FIG. 1 shows a calibration curve of solubility for Ilomastat in pH 7.6 aqueous solution.

FIG. 2 shows the release profile from Ilomastat tablet 1.

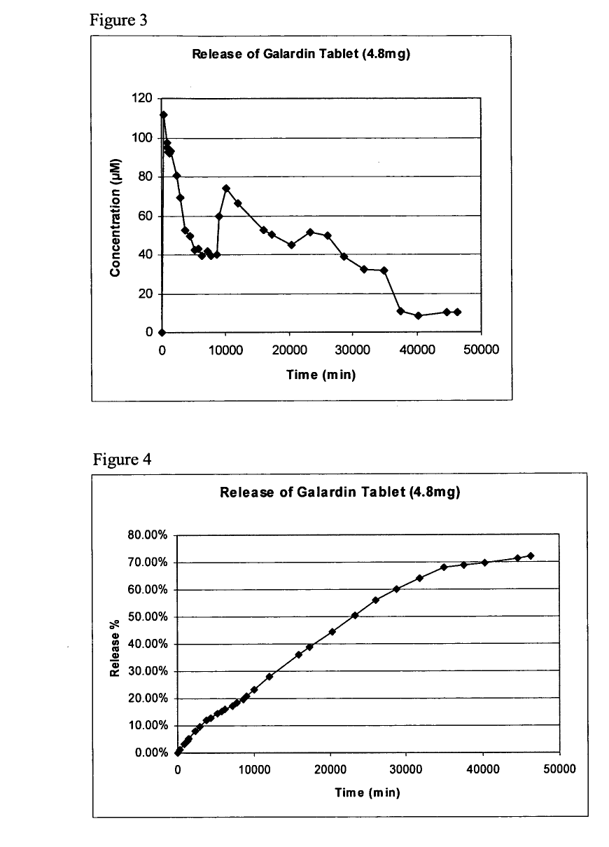

FIG. 3 shows the concentration of Ilomastat in the samples collected from the rig with tablet 1.

FIG. 4 shows the release profile from Ilomastat tablet 2.

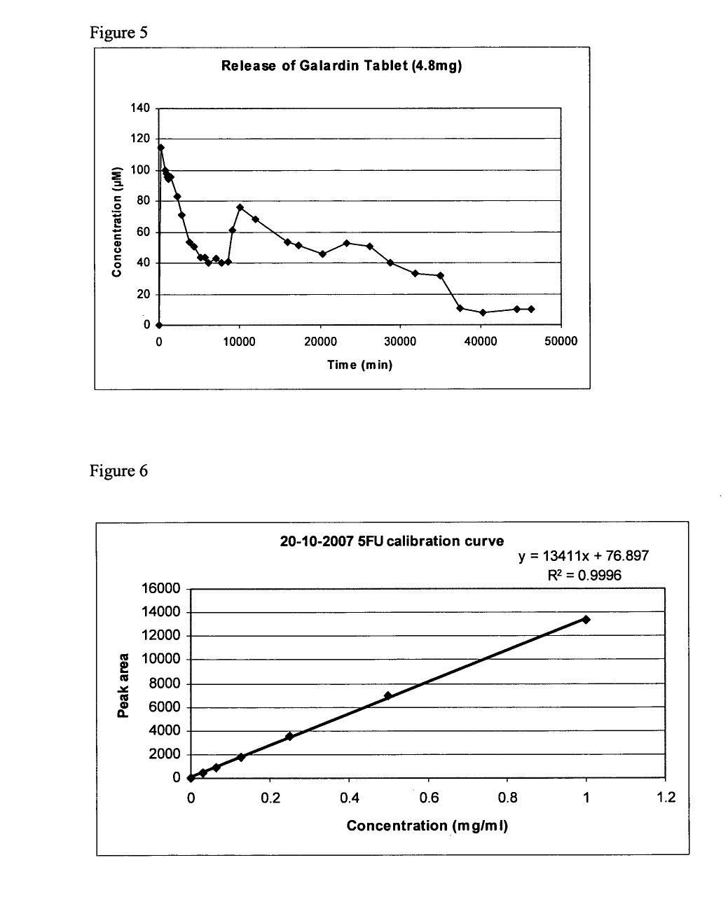

FIG. 5 shows the concentration of Ilomastat in the samples collected from the rig with tablet 2.

FIG. 6 shows a calibration curve of solubility for 5-FU.

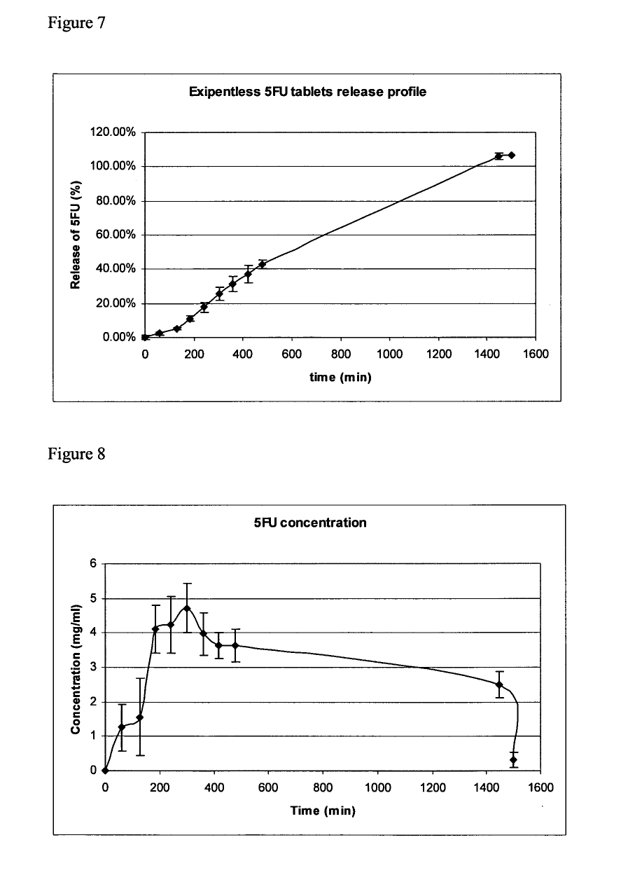

FIG. 7 shows the release profile from the 5-FU tablets.

FIG. 8 shows the concentration of 5-FU in the samples collected from the rig.

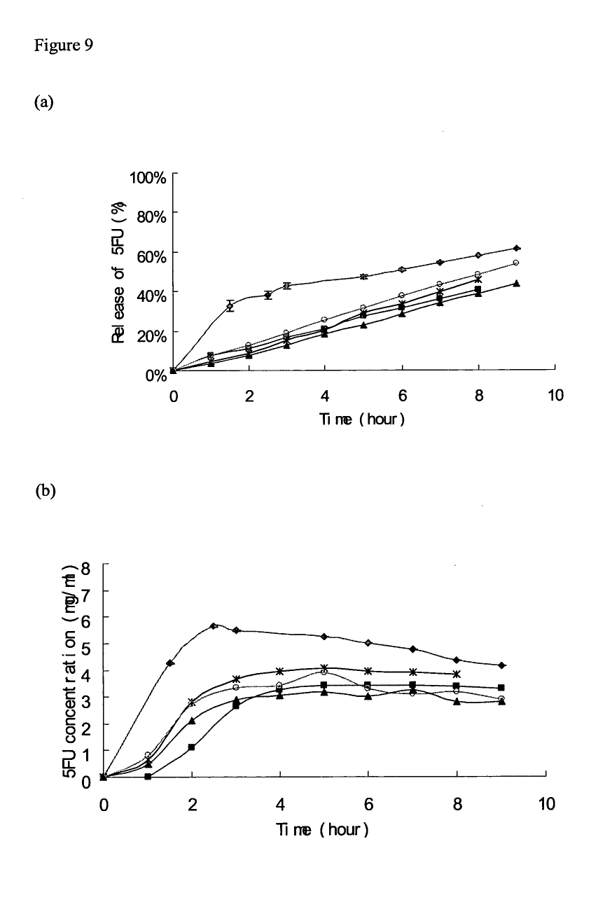

FIG. 9 shows the cumulative release (a) and the concentration (b) of 5-FU released from excipient-free tablets under various conditions. The release profiles show ♦ Tablets in 50 μl chamber, ▪ Tablets at the centre of 200 μl chamber, ▴ Tablets placed in 200 μl chamber closed to the in-going tube, ◯ Tablets placed in 200 μl chamber closed to the out going tube, and * Tablets at the side of 200 μl chamber.

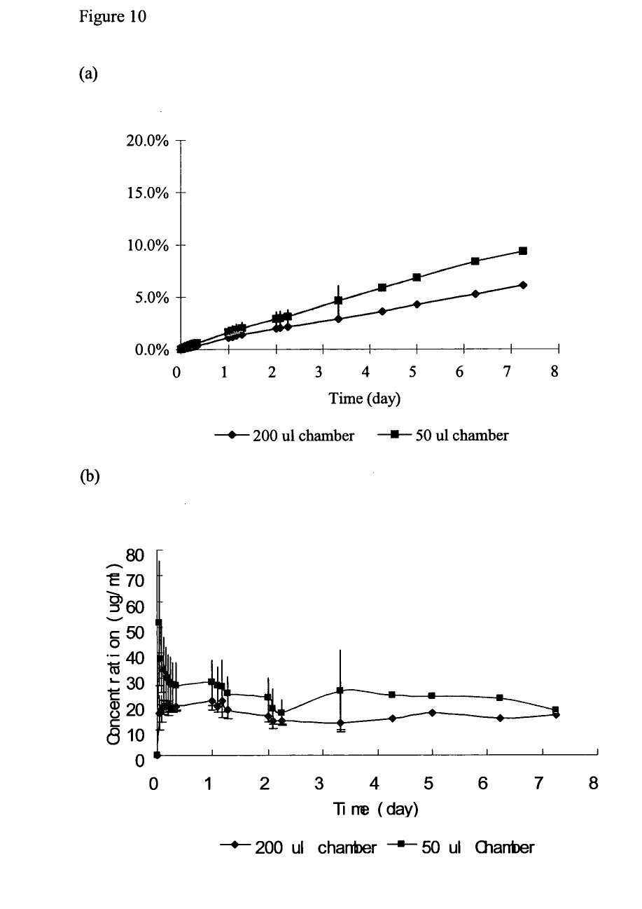

FIG. 10 shows the cumulative release (a) and the concentration (b) of triamcinolone released from excipient free tablets.

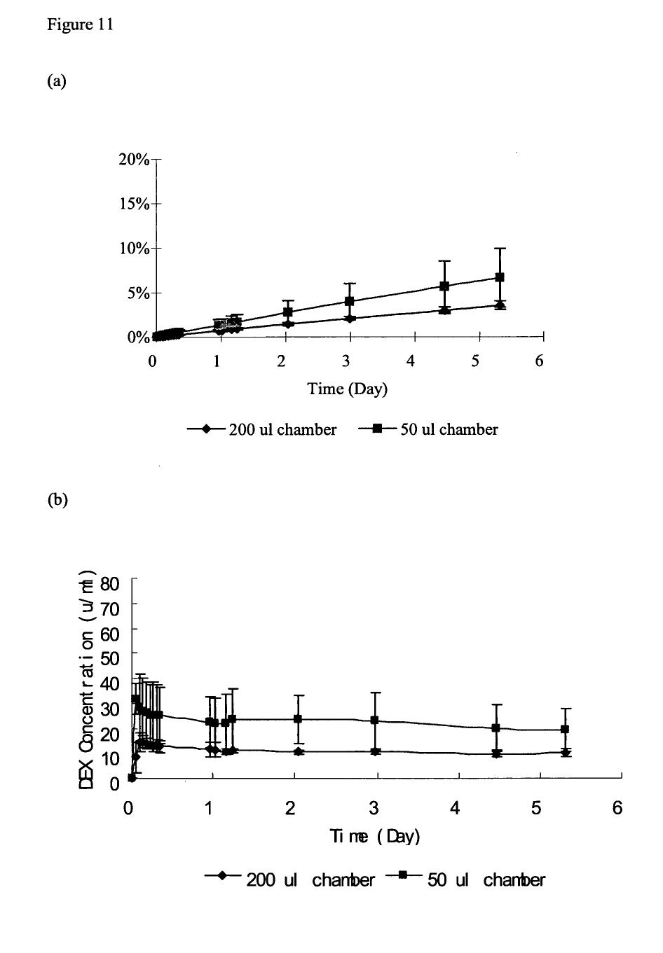

FIG. 11 shows the cumulative release (a) and the concentration (b) of dexamethasone released from excipient free tablets.

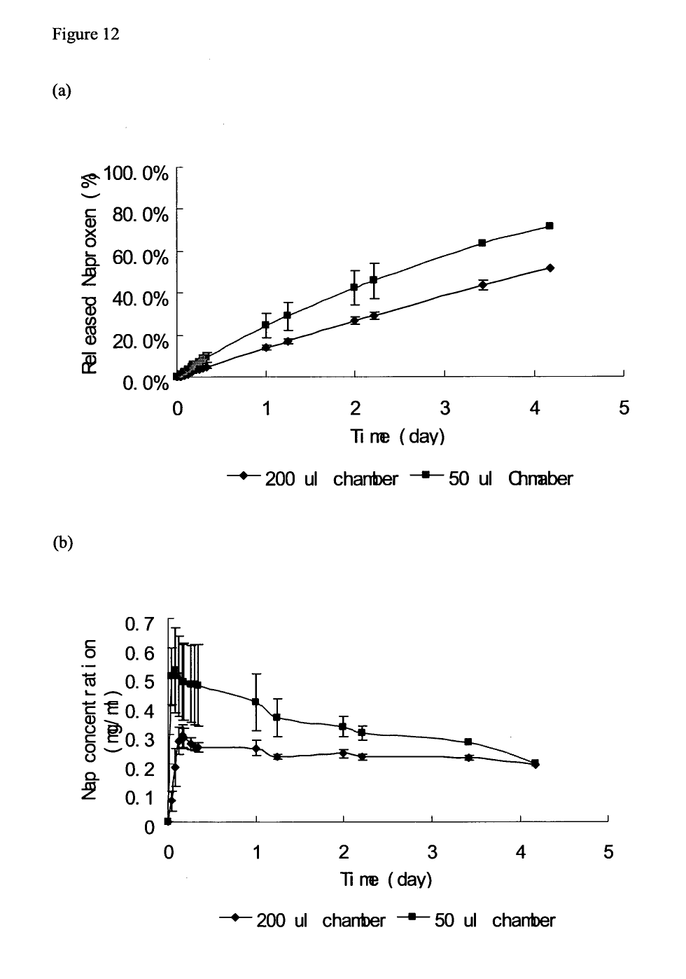

FIG. 12 shows the cumulative release (a) and the concentration (b) of naproxen released from excipient free tablets.

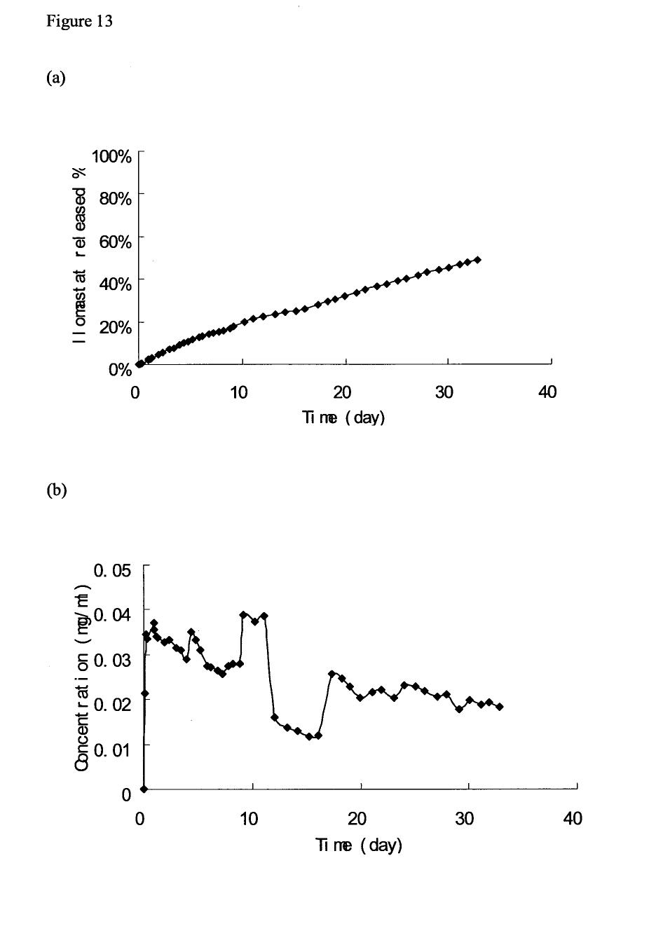

FIG. 13 shows the cumulative release (a) and the concentration (b) of ilomastat released from excipient free tablets in a 200 μl flow dissolution rig.

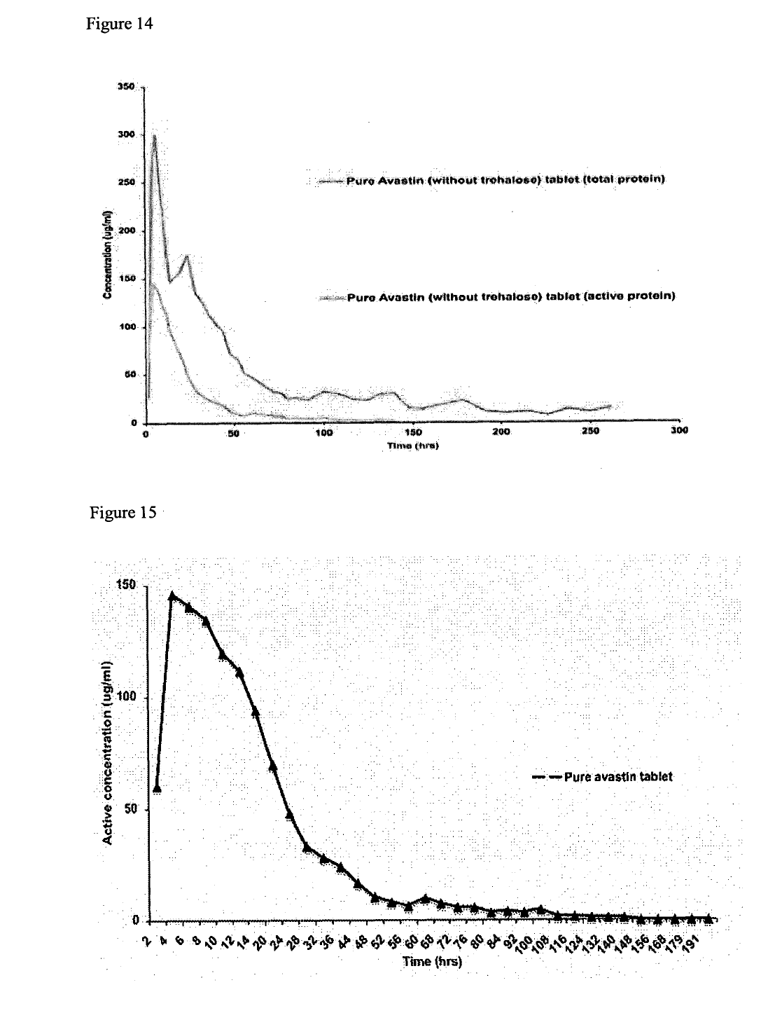

FIG. 14 shows the release profile and retention of activity of bevacizumab from substantially excipient-free tablets.

FIG. 15 shows the ‘active protein’ data of FIG. 14 with actual data points plotted.

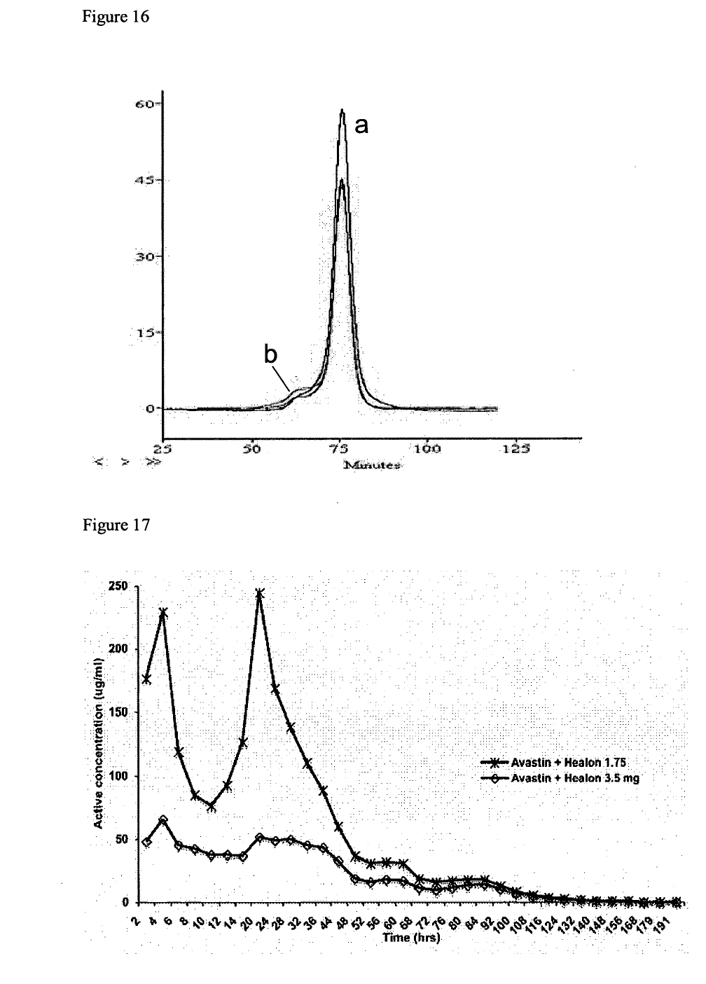

FIG. 16 shows the size exclusion chromatography trace of bevacizumab from excipient free tablets, compared to that obtained from the commercial injectable product Avastin.

FIG. 17 shows the release profile of bevacizumab from tablets according to the invention and containing hyaluronic acid as an excipient.

### EXAMPLES

Solubility experiments suggested the possibility that a therapeutic dose of Ilomastat and other MMP inhibitors can be achieved by slow dissolution of a solid tablet form of Ilomastat or other MMP inhibitors. It was established that compared to simple injections where clearance occurs in less than 5 minutes in an in vitro flow cell, prolonged release could be achieved with Ilomastat in the tablet form. A clinically validated in vivo model of GFS was then used to examine the effects of prolonged release at the site of surgery over different time points up to a period of 30 days. For GFS, Ilomastat has not been found to be toxic; however, the teaching is applicable to a variety of MMP inhibitors and other substantially water insoluble therapeutic agents.

Materials and Methods

Flow System

To obtain some indication of release kinetics, flow rigs of 50-200 μl capacity were used to model the bleb. An Ilomastat tablet (one tablet per rig) was placed into the flow chamber. Two tubes are connected to each rig: one was connected to a peristaltic pump to introduce an aqueous solution and the other tube allowed the removal of the solution out of the rig. Flow rates were used to model the flow of the aqueous solution into and out from the subconjunctival space to the scleral veins. Samples were collected as the solution flowed from the rig to determine the concentration of Ilomastat in this slow release system.

A range of flow rates was used in the rig experiments; however, in most of the experiments a flow rate of 2 μl/min was used to simulate the aqueous flow rate in the bleb. To further simulate the actual conditions in the eye, the aqueous solution used was maintained at pH 7.4-7.6 (as this is the pH of normal human aqueous humor) and the temperature was maintained at 37° C. The aqueous solution that was prepared using Oxoid® Phosphate Buffered Saline Tablets (one tablet for every 100 ml of de-ionized water). The PBS tablets were dissolved in de-ionized water and the pH was adjusted to 7.6. The aqueous solution was kept at 37° C.

Tablet Fabrication

A tablet punch and die was used and solid Ilomastat was placed in the die and the punch was fitted. The solid Ilomastat was accurately weighed prior to the placement in the die. The fitted punch-die was then placed into a tablet compressor and pressed to a pressure of 5 bars for about ten seconds.

HPLC Method

Several reverse phase columns and mobile phases were evaluated to determine the optimal conditions required for HPLC separation of Ilomastat. It was found that a C-18 column (SIGMA) and a 25% acetonitrile aqueous mobile phase gave good base line resolution. The mobile phase was prepared as follows. To make 1000 ml of buffer 1.54 gm of ammonium acetate (Fluka), 6 ml of triethylamine 99.5% (Sigma Aldrich), ˜950 ml de-ionized water were mixed and then approximately 10 ml of acetic acid 100% (Analar BDH) was added to adjust the pH of the buffer to 5.0±0.1. When the pH was adjusted, de-ionized water was added to make the buffer volume of 1000 ml. Aliquots of each sample (0.1 ml) were transferred to HPLC vials that were then placed in the HPLC auto-sampler. The mobile phase eluted at 1 ml/min and the UV detector was set to 280 nm to determine the concentration of an Ilomastat solutions (Galardy et al. 1994b). Three injections (10 μl each) for each time point were evaluated. A computer was connected with the UV detector and with the use of the programme Chrom+, the peak area was analyzed to determine the amount of Ilomastat. The surface of the peak represents the concentration of Ilomastat in the tested solution. The average of the three measurements was used to determine the amount of Ilomastat.

Sterilization of the Tablet with Gamma Radiation

Following the regulations of European and American Pharmacopoeia, it is necessary the final dosage forms of the administered drugs to be sterile. Since tablet fabrication was not conducted in aseptic conditions and since sterile Ilomastat is not commercially available it was necessary to sterilise the Ilomastat tablets using gamma radiation. Gamma radiation is widely used as it has significant advantages including better assurance of product sterilization than aseptic processing and filtration, is penetrating into final fabricated objects, is a low temperature process and has a simple validation process. Also there are no residues which must be removed as for example with ethylene oxide sterilisation. One potential disadvantage is that gamma radiation can initiate chemical reactions that can result in the modification of chemical structure within the sample. Generally, a 25 kGy dose is needed to achieve the minimum sterility assurance level of SAL=10⁻⁶ (the probability is one in a million the item to be non-sterile after the process). Lower doses may be validated using appropriate sterility tests. Under the regulations of European and American Pharmacopoeia, a 25 KGy dose of radiation ensures sterilization (2000a; 2000b). In co-operation with Cranfield University in the UK a Cobalt 60 gamma radiation source was utilized. This is considered suitable to sterilise drugs and biomaterials by irradiation. Ilomastat was thus irradiated as an unprocessed powder and as a fabricated tablet. As the Cobalt 60 gamma source applies about 4500 KGy radiation per hour, the samples were left in the Cobalt 60 panoramic chamber for about 5 hours and 35 minutes in order to obtain the 25 kGys exposure.

In Vitro Experiments

1. Human Tenon's Fibroblasts (HTF)

Human Tenon's fibroblasts (HTF) were used for in vitro cultures. These cells are involved in subconjunctival scarring. The process of HTF isolation and proliferation was performed by using 0.5 cm³ tissue explants from donor eyes obtained from Moorfields Hospital Eye Bank under the Tenets of the Declaration of Helsinki (1989). Explants of 0.5 cm³ were kept for two hours in the bottom of 25 cm³ flasks, with a coverslip placed over them. Each flask contained 5 ml of normal culture medium consisting of Dulbecco's modified Eagle's Medium (DMEM) with 10% fetal calf serum, 2 mM L-glutamine, 100 U/ml penicillin, 50 mg/ml gentamicin, 100 μg/ml streptomycin and 0.25 μg/ml amphotericin. The flasks were placed in incubators at 37° C. and 5% humidified CO₂ in air. The culture medium was changed every 3 days and when they became confluent, usually within one month, they were passaged into new flasks for direct experimental use or were stored in liquid nitrogen.

2. Passaging and Maintenance of Cell Cultures

After the HTFs reached confluence, the culture medium was aspirated and the monolayer was washed with 1 ml of trypsin 1× (Gibco) and the trypsin was quickly aspirated for about 15 seconds. Next, 2 ml of trypsin 1× (Gibco) were added to each flask and HTFs were detached from the flasks by incubation for 2 minutes at 37° C. and 5% humidified CO₂ in air. After confirming by phase contrast microscopy using a Leica microscope with ×10 magnification that the cells had been detached from the bottom of the flask and that they had obtained a round shape, 2 ml of cell culture medium were added to neutralise the trypsinisation. The cell suspension was transferred to a 15 ml centrifuge tube (STARLAB GMBH) and was centrifuged at 1600 rpm for 5 minutes. The cell pellet was then resuspended in 10 ml of cell culture medium and was divided into 4 different 75 cm³ flasks (1:4 expansion). In each flask 7.5 ml of cell culture medium were added. Flasks were placed in incubators at 37° C. and 5% humidified CO₂ in air and the culture media was changed every three days. The time that was required from passage to passage in order to reach confluence was 1 week on average.

3. Preparation of the Collagen Gels (in vitro Contraction Model)

With a Neubauer plate, 6.2×10⁴ HTF were counted and then were resuspended in 170 μl FBS in a 50 ml universal tube. Concentrated medium (160 μl) was added (stock solution consisting of 3.5 ml DMEM (×10 stock), 0.35 ml glutamine (2 mM stock) and 0.9 ml sodium bicarbonate (7.5% stock). 830 μl of First Link type I collagen solution collagen (stock 2.2 mg/ml in 0.6% acetic acid) were then added and the solution was mixed by swirling to avoid air bubbles. Sterile 1M NaOH (75-80 μl) was rapidly added to change the acidic pH of the solution. This caused the solution to turn a pink colour without reverting back to yellow. Quickly 150 μl collagen gel solution was cast into the wells of Mattek dishes making sure to cast the gel to the edges of the central groove using the pipette tip. Creation of air bubbles when ejecting the gel from the tip should be avoided. If air bubbles were formed, they were aspirated out. Usually, from a 1.2 ml gel suspension, 6 gels can easily be cast. Following this process, the wells of Makket dishes with the gels were placed in incubator to set for at least 10-15 min (up to 30 mins). Gels were detached from the edges of the central groove using yellow tips and excess unpolymerised solution was aspirated off. Two milliliters of cell culture medium were added and the dishes were placed in incubators at 37° C. and 5% humidified CO₂ in air; the medium was replaced every 3 days.

4. Preparation of Media with Ilomastat

Generally solid Ilomastat is diluted into DMSO before being added into the media, however in this experiment Ilomastat could be directly dissolved in the normal media without DMSO. Media and solid Ilomastat that had been sterilised, and media and solid non-irradiated Ilomastat were placed in different 50 ml universal tubes and stirred for about 5-6 hours. The concentration of both samples was then confirmed by HPLC.

5. In vitro Evaluation of Ilomastat Activity

The inhibitory effect of non-irradiated Ilomastat on HTF contraction of collagen I gels is known. To compare with irradiated Ilomastat, experiments were conducted with three different treatment groups of collagen gels. Each treatment group had 3 collagen I gels with HTFs. The gels of the first group were treated with media without Ilomastat (negative control), the gels of the second group were treated with media with non-irradiated Ilomastat (positive control) and the gels of the third group were treated with media with irradiated Ilomastat.

In a second in vitro experiment the inhibitory effect of the irradiated Ilomastat tablet dissolved directly in normal media was compared with the non-irradiated Ilomastat powder dissolved initially in DMSO and then in normal media. This experiment was conducted to determine if the tablet fabrication process results in solid state changes such as crystallisation which could lead to reduced effectiveness of Ilomastat.

The inhibitory effect of Ilomastat was determined by measuring the contraction of the collagen gels. Photographs of the gels were obtained daily. The % contraction was determined using the software called Image J. The media of the treated gels then stored at −70° C. for future zymographic analysis in order to test the levels of active MMPs.

In Vivo Experiment

1. Experimental Design

A random, one block study design was performed, with 4 rabbits undergoing glaucoma drainage surgery to the left eye. Animals were observed for a period of 30 days. The experiment was performed as a randomised, blind, controlled study with masked observers. One observer was used to assess clinical data.

2. Animals

Four Female New Zealand White Rabbits (Harlan UK Ltd; c. 2-2.2 kg, 12-14 weeks old) were used. Animals were housed in the BRU Unit of Ophthalmology and were allowed an acclimatisation period of 7 days, as is normally required.

3. Treatment Regimen

Animals were randomly assigned to either of two groups, as shown in Table 1. Animals in Group A received the Ilomastat excipient free tablet (also referred to as a pellet) and Group B received the ethylcellulose tablet which was used as the control. Ethylcellulose is an excipient that does not dissolve in aqueous solution and does not have any known inhibitory activity against MMP's. The size of the ethylcellulose tablet remained unchanged during the 30 day period of the in vivo experiment. The control pellet was the same size as the Ilomastat pellet in order to determine if the biological activity of Ilomastat itself maintained the bleb and its functionality rather than the simple placement of an inert ethylcellulose tablet.

TABLE 1
TREATMENT GROUPS

| | | Tablet | | Control | |
|---|---|---|---|---|---|
| Group # | Treatment | characteristics | Schedule | eye (right) | Study End |
| A | Ilomastat | Weight: 2.1-2.3 mg | Placement | No | Day 30 - all rabbit |
| (3 rabbits) | tablet | Diameter: 3 mm | of one pellet | treatment | eyes to histology |
| | | Thickness: 0.4 mm | in the left eye | | |
| | | | during GFS | | |
| B | Ethylcellulose | Weight: 1.5 mg | Placement | No | Day 30 - all rabbit |
| (1 rabbits) | tablet | Diameter: 3 mm | of one pellet | treatment | eyes to histology |
| | | Thickness: 0.4 mm | in the left eye | | |
| | | | during GFS | | |

Either an Ilomastat or an ethylcellulose tablet was placed subconjunctivally into the left eye just before conjunctival closure at the end of GFS.

4. Glaucoma Filtering Operation—Model of Glaucoma Filtration Surgery

Surgery is carried out using a standard method that has been thoroughly described in the literature. The consistency of the surgical procedure and its use in the rabbit allows for approximate comparisons with previous studies. This is particularly important since this model is clinically validated and this surgical procedure is in wide clinical use.

5. Collection of Samples

At the end of the experiment on day 30, aqueous humor, vitreous and blood were collected for Ilomastat detection on HPLC.

Results

Calibration Curve

A calibration curve for Ilomastat at pH 7.6 in aqueous solution without DMSO is shown in FIG. 1. The curve was generated by measurement of Ilomastat as it eluted from the HPLC column (C 18) using the mobile phase as described above and a UV detector (280 nm) with the software called Chrom+.

The curve was created as follows. Ilomastat (0.3885 mg) (Caldiochem, purity>95%) was dissolved in 7.6 pH aqueous solution (10 ml) to a give a stock solution at a concentration of 100 μM. The stock solution was then diluted in individual containers to give six other solutions with the following concentrations: 80 μM, 60 μM, 40 μM, 20 μM, 10 μM and 5 μM. Each solution was then evaluated three times by HPLC and the absorbance was determined. The Ilomastat peak was detected at approximately 6-8 min after the injection. The average calibration curve obtained is shown in the FIG. 1.

Ilomastat Tablet Release

The overall aim was to determine if placing a small tablet made of compressed pure Ilomastat in the subconjunctival space after glaucoma filtration surgery could result in slow release of Ilomastat in the aqueous humor. As Ilomastat is a very expensive compound, experience was obtained in small tablet fabrication using other compounds such as 5-FU prior to the formation of the Ilomastat tablets. Three excipient free Ilomastat tablets were fabricated using 6.5 mg, 5.6 mg and 3.2 mg of solid Ilomastat. A standard tablet punch and die and a press with an applied a pressure of five bars were used. The first tablet had a diameter of 3 mm, a thickness of 0.87 mm and a weight of 4.8 mg. The second tablet had the same diameter, a thickness of 0.62 mm and a weight of 4.1 mg. The third tablet had diameter of 3 mm, a thickness of 0.4 mm and a weight of 2.3 mg. Small amounts of Ilomastat remained on the surface of the punch and die. The quantity of the Ilomastat that was used for the first tablet fabrication was based on the hypothesis that in every time point during the period of thirty days, Ilomastat would maintain the theoretical maximum dissolution in the aqueous solution (about 100 μM).

After the placement of each tablet into the rig, 7.6 pH aqueous solution was pumped into the rig. The flow rate was set in 2 μl/min, similar to the flow rate of aqueous humour through the trabecular meshwork. Liquid samples were collected after exiting the rig. The samples after filtration were then analysed by HPLC and the concentration of Ilomastat was determined using the calibration curve.

The data from tablets A and B were used to graphically show the release profiles for each tablet (FIGS. 2, 3, 4 and 5)

Fabrication of the Ilomastat Tablet to be Used in the in vivo Experiments

As the two tablets tested were found not to dissolve completely after 30 days in the flow rig, the inventors attempted to create a softer tablet using 2.3 mg of Ilomastat. We placed this tablet in a flow rig of 200 p1 capacity, the system was set in flow rate 2 μl/min and the release profile of this tablet is shown in the Table 4.

TABLE 2
Ilomastat tablet 1

| | Galardin Tablet A without excipient (W = 4.8 mg) started on 14 Dec. 2006 HPLC results | | | | | | | | | |
|---|---|---|---|---|---|---|---|---|---|---|
| | | | | | | | | | Released | |
| | | time | | | | | Volumn | Concentration | Amount | |
| Collect point | No. | point | Peak Area | | | AVR PA | (ml) | (μM) | (mg) | Release % |
| 19.00 14 Dec. 2006 | 0 | 0 | | | | | | 0 | 0 | 0.00% |
| 23.40 14 Dec. 2006 | 1 | 280 | 157.488 | 155.957 | 156.809 | 156.7513333 | 1.2001 | 114.7491472 | 0.05350051 | 1.11% |
| 9.30 15 Dec. 2006 | N1 | 870 | 137.229 | 135.642 | 136.502 | 136.4576667 | 2.592 | 99.91459552 | 0.100613198 | 3.21% |
| 11.40 15 Dec. 2006 | 2 | 1000 | 134.968 | 132.079 | 133.747 | 133.598 | 0.629 | 97.82419591 | 0.023904956 | 3.71% |
| 13.30 15 Dec. 2006 | 3 | 1110 | 129.648 | 130.721 | 130.331 | 130.2333333 | 0.5332 | 95.36464425 | 0.019754614 | 4.12% |
| 15.30 15 Dec. 2006 | 4 | 1230 | 129.561 | 128.325 | 129.147 | 129.011 | 0.5724 | 94.47112573 | 0.021008243 | 4.56% |
| 19.30 15 Dec. 2006 | 5 | 1470 | 131.138 | 132.783 | 129.365 | 131.0953333 | 0.9864 | 95.99476121 | 0.036786767 | 5.32% |
| 9.35 16 Dec. 2006 | 6 | 2315 | 112.444 | 113.384 | 113.338 | 113.0553333 | 4.1481 | 82.80762671 | 0.133447542 | 8.10% |
| 19.30 16 Dec. 2006 | 7 | 2910 | 97.03 | 97.095 | 98.225 | 97.45 | 2.9284 | 71.4002193 | 0.081230844 | 9.80% |
| 9.45 17 Dec. 2006 | 8 | 3765 | 73.847 | 73.202 | 72.517 | 73.18866667 | 4.7233 | 53.66532651 | 0.098475984 | 11.85% |
| 19.30 17 Dec. 2006 | 9 | 4350 | 69.816 | 67.342 | 69.007 | 68.72166667 | 2.6526 | 50.39997563 | 0.051938944 | 12.93% |
| 9.30 18 Dec. 2006 | 10 | 5190 | 59.512 | 59.458 | 58.878 | 59.28266667 | 3.89 | 43.50012183 | 0.065740212 | 14.30% |
| 17.45 18 Dec. 2006 | 11 | 5685 | 58.327 | 60.744 | 59.668 | 59.57966667 | 2.4366 | 43.7172271 | 0.041383562 | 15.16% |
| 17.45 19 Dec. 2006 | 13 | 6180 | 55.1 | 54.563 | 54.488 | 54.717 | 2.0125 | 40.1626462 | 0.031401416 | 15.82% |
| 9.40 20 Dec. 2006 | 14 | 7135 | 58.549 | 57.778 | 58.256 | 58.19433333 | 4.264 | 42.70455653 | 0.070742831 | 17.29% |
| 19.10 20 Dec. 2006 | 15 | 7705 | 54.922 | 55.193 | 54.477 | 54.864 | 2.7983 | 40.27010234 | 0.043779221 | 18.20% |
| 9.40 21 Dec. 2006 | 16 | 8575 | 55.348 | 55.694 | 55.868 | 55.63666667 | 4.151 | 40.83491715 | 0.06585298 | 19.57% |
| 18.15 21 Dec. 2006 | 17 | 9090 | 83.965 | 83.557 | 83.618 | 83.71333333 | 2.1473 | 61.3587963 | 0.051187106 | 20.64% |
| 10.15 22 Dec. 2006 | 18 | 10050 | 103.506 | 104.524 | 103.178 | 103.736 | 4.1123 | 75.99524854 | 0.121412179 | 23.17% |
| 18.15 23 Dec. 2006 | 19 | 11970 | 94.154 | 93.2 | 91.605 | 92.98633333 | 8.7515 | 68.13730507 | 0.231663958 | 28.00% |
| 12.15 26 Dec. 2006 | 20 | 15930 | 72.826 | 73.267 | 72.786 | 72.95966667 | 18.8311 | 53.49792885 | 0.391384553 | 36.15% |
| 11.45 27 Dec. 2006 | 21 | 17340 | 69.403 | 70.947 | 69.854 | 70.068 | 6.7603 | 51.38413743 | 0.134954094 | 38.96% |
| 13.30 29 Dec. 2006 | 22 | 20325 | 62.785 | 62.415 | 63.097 | 62.76566667 | 14.4484 | 46.04617446 | 0.258466543 | 44.35% |
| 14.55 31 Dec. 2006 | 23 | 23290 | 71.743 | 71.822 | 71.599 | 71.72133333 | 14.0847 | 52.59271442 | 0.287782387 | 50.34% |
| 14.00 02 Jan. 2007 | 24 | 26115 | 68.958 | 69.033 | 69.623 | 69.20466667 | 13.4965 | 50.75304581 | 0.266118026 | 55.89% |
| 9.55 04 Jan. 2007 | 25 | 28750 | 54.295 | 54.414 | 53.755 | 54.15466667 | 12.9323 | 39.75158382 | 0.19971985 | 60.05% |
| 13.00 06 Jan. 2007 | 26 | 31815 | 44.125 | 44.864 | 45.289 | 44.75933333 | 15.0127 | 32.8836501 | 0.191791717 | 64.04% |
| 18.00 08 Jan. 2007 | 27 | 34995 | 43.208 | 43.358 | 43.558 | 43.37466667 | 15.2311 | 31.87146686 | 0.188592468 | 67.97% |
| 12.15 10 Jan. 2007 | 28 | 37530 | 14.723 | 14 | 14.022 | 14.24833333 | 11.4824 | 10.58028752 | 0.047197736 | 68.95% |
| 10.25 12 Jan. 2007 | 29 | 40300 | 10.038 | 10.702 | 10.739 | 10.493 | 12.6964 | 7.835160819 | 0.038647333 | 69.76% |
| 10.00 15 Jan. 2007 | 30 | 44595 | 12.813 | 13.096 | 13.132 | 13.01366667 | 20.0826 | 9.677753411 | 0.075506704 | 71.33% |
| 14.40 16 Jan. 2007 | 31 | 46315 | 12.744 | 12.918 | 13.31 | 12.99066667 | 8.0509 | 9.660940546 | 0.030217245 | 71.96% |

| | Galardin Tablet B without excipient (W = 4.1 mg) started on 17 Jan. 2006 HPLC results | | | | | | | | | |
|---|---|---|---|---|---|---|---|---|---|---|
| | | | | | | | | | Released | |
| | | time | | | | | Volumn | Concentration | Amount | |
| Collection point | No. | point | Peak Area | | | AVR PA | (ml) | (μM) | (mg) | Release % |
| 17 Jan. 2007 12:10 | 0 | 0 | | | | | | 0 | 0 | 0.00% |
| 17 Jan. 2007 14:15 | 1 | 125 | 76.724 | 76.738 | 77.774 | 77.07866667 | 0.2353 | 55.16239618 | 0.005042618 | 0.12% |
| 17 Jan. 2007 16:20 | 2 | 250 | 124.471 | 124.647 | 124.983 | 124.7003333 | 0.2453 | 88.77689937 | 0.008460354 | 0.33% |
| 17 Jan. 2007 18:40 | 3 | 390 | 121.179 | 121.461 | 121.376 | 121.3386667 | 0.2683 | 86.40401402 | 0.009006284 | 0.55% |
| 18 Jan. 2007 10:15 | 4 | 1325 | 134.017 | 134.277 | 134.948 | 134.414 | 1.8601 | 95.63344392 | 0.069109398 | 2.23% |
| 18 Jan. 2007 12:20 | 5 | 1450 | 128.249 | 128.879 | 129.151 | 128.7596667 | 0.241 | 91.64224371 | 0.008580326 | 2.44% |
| 18 Jan. 2007 14:55 | 6 | 1605 | 122.177 | 123.375 | 123.525 | 123.0256667 | 0.318 | 87.59480953 | 0.010821726 | 2.71% |
| 18 Jan. 2007 18:10 | 7 | 1800 | 121.623 | 122.639 | 122.078 | 122.1133333 | 0.3767 | 86.95082469 | 0.012725075 | 3.02% |
| 19 Jan. 2007 10:00 | 8 | 2750 | 118.387 | 117.594 | 118.36 | 118.1136667 | 1.897 | 84.127597 | 0.062000735 | 4.53% |
| 19 Jan. 2007 19:10 | 9 | 3300 | 118.88 | 120.321 | 120.56 | 119.9203333 | 1.0905 | 85.40286111 | 0.036181712 | 5.41% |
| 20 Jan. 2007 11:40 | 10 | 4286 | 113.81 | 114.048 | 114.532 | 114.13 | 1.9651 | 81.31566316 | 0.06207974 | 6.93% |
| 20 Jan. 2007 21:20 | 11 | 4866 | 111.149 | 111.503 | 111.717 | 111.4563333 | 1.1597 | 79.42841345 | 0.035785951 | 7.80% |
| 21 Jan. 2007 11:45 | 12 | 5726 | 104.638 | 104.145 | 103.55 | 104.111 | 1.7169 | 74.24359427 | 0.049521639 | 9.01% |
| 21 Jan. 2007 23:05 | 13 | 6286 | 126.784 | 126.61 | 127.319 | 126.9043333 | 1.1772 | 90.33262747 | 0.041312923 | 10.02% |
| 22 Jan. 2007 09:40 | 14 | 6921 | 119.29 | 120.395 | 120.544 | 120.0763333 | 1.096 | 85.51297617 | 0.036411083 | 10.90% |
| 22 Jan. 2007 20:05 | 15 | 7526 | 111.619 | 111.656 | 111.987 | 111.754 | 1.0547 | 79.63852615 | 0.032631962 | 11.70% |
| 23 Jan. 2007 10:30 | 16 | 8387 | 99.59 | 98.474 | 99.683 | 99.249 | 1.5183 | 70.81167502 | 0.041768943 | 12.72% |
| 23 Jan. 2007 17:30 | 17 | 8807 | 97.844 | 97.385 | 98.147 | 97.792 | 0.7384 | 69.78322863 | 0.020018603 | 13.21% |
| 24 Jan. 2007 10:25 | 18 | 9822 | 95.043 | 95.341 | 95.806 | 95.39666667 | 1.396 | 68.09244488 | 0.036929665 | 14.11% |
| 24 Jan. 2007 21:00 | 19 | 10454 | 92.692 | 92.532 | 92.841 | 92.68833333 | 0.8332 | 66.18072516 | 0.021422582 | 14.63% |
| 25 Jan. 2007 09:29 | 20 | 11203 | 99.118 | 99.057 | 99.257 | 99.144 | 1.0931 | 70.73755912 | 0.030040073 | 15.36% |
| 25 Jan. 2007 19:05 | 21 | 11779 | 100.226 | 100.796 | 100.72 | 100.5806667 | 0.8713 | 71.7516529 | 0.024287938 | 15.95% |
| 26 Jan. 2007 10:20 | 22 | 12694 | 101.087 | 101.081 | 100.8 | 100.9893333 | 1.5272 | 72.0401167 | 0.04274264 | 17.00% |
| 26 Jan. 2007 18:50 | 23 | 13204 | 140.128 | 140.666 | 141.232 | 140.6753333 | 0.8061 | 100.0531046 | 0.031333616 | 17.76% |
| 27 Jan. 2007 19:10 | 24 | 14664 | 133.228 | 133.705 | 136.782 | 134.5716667 | 2.0833 | 95.74473542 | 0.077492155 | 19.65% |
| 28 Jan. 2007 15:20 | 25 | 15874 | 139.667 | 139.716 | 140.24 | 139.8743333 | 1.9183 | 99.48770617 | 0.074144163 | 21.46% |
| 29 Jan. 2007 15:00 | 26 | 17294 | 57.499 | 57.445 | 57.715 | 57.553 | 2.6583 | 41.37989694 | 0.04273507 | 22.50% |
| 30 Jan. 2007 18:00 | 27 | 18914 | 49.137 | 49.309 | 49.563 | 49.33633333 | 3.0499 | 35.58003341 | 0.042158289 | 23.53% |
| 31 Jan. 2007 17:30 | 28 | 20324 | 46.969 | 46.287 | 45.8 | 46.352 | 2.4316 | 33.47349474 | 0.031621627 | 24.30% |
| 01 Feb. 2007 18:45 | 29 | 21841 | 41.858 | 42.031 | 41.797 | 41.89533333 | 2.7759 | 30.32768641 | 0.032706504 | 25.10% |
| 02 Feb. 2007 14:15 | 30 | 23011 | 42.755 | 42.323 | 42.551 | 42.543 | 2.2261 | 30.78485212 | 0.026623967 | 25.75% |
| 03 Feb. 2007 21:45 | 31 | 24901 | 93.056 | 92.924 | 92.431 | 92.80366667 | 3.3564 | 66.26213501 | 0.086403266 | 27.86% |
| 04 Feb. 2007 21:50 | 32 | 26346 | 88.894 | 88.676 | 88.447 | 88.67233333 | 2.776 | 63.34596833 | 0.068317107 | 29.52% |
| 05 Feb. 2007 15:25 | 33 | 27401 | 82.583 | 82.637 | 82.669 | 82.62966667 | 1.9655 | 59.08065693 | 0.045113798 | 30.62% |
| 06 Feb. 2007 14:02 | 34 | 28758 | 72.349 | 72.755 | 72.87 | 72.658 | 2.5736 | 52.04199901 | 0.05203386 | 31.89% |
| 07 Feb. 2007 17:45 | 35 | 30421 | 77.961 | 77.421 | 78.448 | 77.94333333 | 3.1176 | 55.77273476 | 0.067551245 | 33.54% |
| 08 Feb. 2007 16:25 | 36 | 31661 | 79.964 | 79.743 | 79.56 | 79.75566667 | 2.4373 | 57.05199878 | 0.054022027 | 34.86% |
| 09 Feb. 2007 20:12 | 37 | 33328 | 73.109 | 73.59 | 72.785 | 73.16133333 | 2.9739 | 52.39728477 | 0.060537735 | 36.33% |
| 10 Feb. 2007 20:02 | 38 | 34758 | 83.51 | 82.953 | 82.943 | 83.13533333 | 2.5168 | 59.4375897 | 0.058116696 | 37.75% |
| 11 Feb. 2007 19:39 | 39 | 36175 | 82.228 | 82.482 | 82.312 | 82.34066667 | 2.4738 | 58.87666173 | 0.05658467 | 39.13% |
| 12 Feb. 2007 15:24 | 40 | 37460 | 78.173 | 78.153 | 78.111 | 78.14566667 | 2.1196 | 55.91555493 | 0.04604448 | 40.25% |
| 13 Feb. 2007 19:00 | 41 | 39110 | 73.434 | 73.821 | 73.954 | 73.73633333 | 2.9937 | 52.80315757 | 0.061412842 | 41.75% |
| 14 Feb. 2007 16:18 | 42 | 40388 | 75.344 | 75.714 | 75.904 | 75.654 | 2.4126 | 54.15677278 | 0.050760878 | 42.99% |
| 15 Feb. 2007 18:15 | 43 | 41945 | 63.16 | 63.386 | 63.451 | 63.33233333 | 2.9028 | 45.45933037 | 0.051266205 | 44.24% |
| 16 Feb. 2007 17:40 | 44 | 43342 | 71.482 | 71.508 | 71.743 | 71.57766667 | 2.2821 | 51.27942872 | 0.045464129 | 45.35% |
| 17 Feb. 2007 19:20 | 45 | 44882 | 67.218 | 67.43 | 67.623 | 67.42366667 | 2.664 | 48.34726242 | 0.050037676 | 46.57% |
| 18 Feb. 2007 14:30 | 46 | 46032 | 68.866 | 68.951 | 69.157 | 68.99133333 | 2.0982 | 49.45382462 | 0.04031232 | 47.55% |
| 19 Feb. 2007 11:25 | 47 | 47347 | 65.282 | 65.489 | 65.722 | 65.49766667 | 2.106 | 46.98776499 | 0.038444497 | 48.49% |

TABLE 4
RELEASE PARAMETERS OF THE 2.3 mg ILOMASTAT TABLET

| | | | | | | | | | | Released | |
|---|---|---|---|---|---|---|---|---|---|---|---|
| | | time | | | | | | Volumn | Concentration | Amount | |
| Collection point | No. | point | Peak Area | | | | AVR PA | (ml) | (μM) | (mg) | Release % |
| 27 May 2007 11:25 | 0 | 0 | | | | | | | 0 | 0 | 0.00% |
| 28 May 2007 13:35 | 1 | 1570 | 118.017 | 117.66 | 117.746 | 117.466 | 117.72225 | 3.1243 | 83.85130938 | 0.101777927 | 4.43% |
| 29 May 2007 16:50 | 2 | 3205 | 114.342 | 114.49 | 114.053 | 114.761 | 114.4115 | 3.2863 | 81.51436437 | 0.104071635 | 8.95% |
| 30 May 2007 13:15 | 3 | 5655 | 113.414 | 113.822 | 113.69 | 113.546 | 113.618 | 2.4501 | 80.9542599 | 0.077057434 | 12.30% |
| 31 May 2007 14:30 | 4 | 7170 | 62.568 | 62.567 | 62.588 | 62.633 | 62.589 | 3.302 | 44.93463683 | 0.057643365 | 14.81% |
| 01 Jun. 2007 09:20 | 5 | 8300 | 77.02 | 77.173 | 76.899 | 76.791 | 76.97075 | 2.2374 | 55.0862215 | 0.047882591 | 16.89% |
| 02 Jun. 2007 16:55 | 6 | 10195 | 75.027 | 75.15 | 75.409 | 75.137 | 75.18075 | 3.6196 | 53.82272182 | 0.075686297 | 20.18% |
| 04 Jun. 2007 08:10 | 7 | 12430 | 79.74 | 78.995 | 79.474 | 78.903 | 79.278 | 4.4702 | 56.71483024 | 0.098495097 | 24.46% |
| 05 Jun. 2007 11:25 | 8 | 14065 | 71.271 | 71.165 | 71.17 | 71.141 | 71.18675 | 3.3518 | 51.00349404 | 0.066415439 | 27.35% |
| 06 Jun. 2007 17:10 | 9 | 15850 | 71.049 | 71.059 | 71.301 | 71.187 | 71.149 | 3.5702 | 50.9768476 | 0.070706045 | 30.42% |
| 07 Jun. 2007 16:15 | 10 | 17235 | 57.555 | 58.416 | 58.827 | 58.799 | 58.39925 | 2.7007 | 41.97723583 | 0.044043437 | 32.34% |
| 08 Jun. 2007 19:20 | 11 | 18980 | 72.262 | 72.248 | 72.361 | 72.499 | 72.3425 | 3.839 | 51.81929837 | 0.07728597 | 35.70% |
| 09 Jun. 2007 12:45 | 12 | 20025 | 73.139 | 72.187 | 72.203 | 72.141 | 72.4175 | 2.0691 | 51.8722383 | 0.041697258 | 37.51% |
| 10 Jun. 2007 13:05 | 13 | 21485 | 55.137 | 56.024 | 55.776 | 55.777 | 55.6785 | 2.92 | 40.05675161 | 0.04544118 | 39.49% |
| 11 Jun. 2007 21:40 | 14 | 23440 | 58.535 | 53.968 | 54.773 | 54.357 | 55.40825 | 3.9206 | 39.86599139 | 0.060722008 | 42.13% |
| 13 Jun. 2007 07:05 | 15 | 25445 | 54.524 | 54.362 | 54.123 | 54.705 | 54.4285 | 4.1503 | 39.17441943 | 0.063164503 | 44.87% |
| 14 Jun. 2007 12:30 | 16 | 27210 | 63.931 | 64.116 | 64.717 | 64.678 | 64.3605 | 3.5123 | 46.185078 | 0.063020858 | 47.61% |
| 15 Jun. 2007 11:35 | 17 | 28605 | 62.099 | 61.387 | 62.312 | 61.232 | 61.7575 | 2.79 | 44.34770947 | 0.048069148 | 49.70% |
| 17 Jun. 2007 16:05 | 18 | 30315 | 57.275 | 57.316 | 57.37 | 57.441 | 57.3505 | 3.4029 | 41.23695913 | 0.054516359 | 52.07% |
| 18 Jun. 2007 12:00 | 19 | 31510 | 49.415 | 49.415 | 49.325 | 48.939 | 49.2735 | 2.4139 | 35.53568151 | 0.033325367 | 53.52% |
| 19 Jun. 2007 15:15 | 20 | 33145 | 62.381 | 62.381 | 62.764 | 62.493 | 62.50475 | 3.3062 | 44.87516764 | 0.057640299 | 56.03% |
| 20 Jun. 2007 15:05 | 21 | 34575 | 57.489 | 57.127 | 58.444 | 58.467 | 57.88175 | 2.86 | 41.61195031 | 0.046235454 | 58.04% |
| 21 Jun. 2007 17:05 | 22 | 36135 | 53.12 | 53.567 | 53.99 | 54.233 | 53.7275 | 3.088 | 38.67960754 | 0.046403461 | 60.06% |
| 22 Jun. 2007 16:15 | 23 | 37525 | 54.112 | 54.232 | 54.767 | 55.001 | 54.528 | 2.731 | 39.24465307 | 0.041638322 | 61.87% |
| 23 Jun. 2007 19:30 | 24 | 39160 | 50.67 | 50.997 | 51.237 | 51.324 | 51.057 | 3.204 | 36.79459307 | 0.045800217 | 63.86% |
| 24 Jun. 2007 14:30 | 25 | 40300 | 43.597 | 44.01 | 44.633 | 44.769 | 44.25225 | 2.273 | 31.99135314 | 0.0282503 | 65.09% |
| 25 Jun. 2007 17:05 | 26 | 41895 | 47.121 | 47.311 | 47.453 | 47.899 | 47.446 | 3.136 | 34.24571187 | 0.041722784 | 66.90% |
| 26 Jun. 2007 09:35 | 27 | 42765 | 49.299 | 48.931 | 49.444 | 49.56 | 49.3085 | 1.731 | 35.56038681 | 0.023914129 | 67.94% |
| 27 Jun. 2007 22:30 | 28 | 44980 | 44.503 | 47.641 | 47.989 | 47.276 | 46.85225 | 4.43 | 33.82660408 | 0.058217446 | 70.47% |

Comparison of Irradiated and Non-Irradiated Ilomastat with HPLC

Implantation of the Ilomastat tablet during glaucoma filtration surgery required that the tablet be sterile. The International Conference on Harmonization (ICH) recommends the use of high-performance liquid chromatography (HPLC), mass spectrometry or gas chromatography to characterize and compare the irradiated product versus the non-irradiated product. Following these guidelines gamma irradiated Ilomastat was dissolved in pH 7.6 aqueous solution and evaluated it by HPLC. The chromatogram for the irradiated Ilomastat was compared with the chromatogram for the non-irradiated Ilomastat. The chromatogram of the irradiated Ilomastat has displayed an extra peak representing compared to total Ilomastat, 0.25% trace products being formed after irradiation. This meets the criteria for both the American and European Pharmacopoeias.

Stability of Ilomastat Tablet

A solution of Ilomastat in DMSO or water at a concentration of 0.1 mM decomposes 1% per month at 4° C. and at 37° C. this increases to 1% per day (Caldiochem data). No data have been published that describe the stability of Ilomastat as a solid tablet when left at 37° C. in a moist environment for several days. An Ilomastat tablet was evaluated for possible decomposition while in an aqueous environment at 37° C. for 30 days. After collecting samples from the second tablet, the inventors removed the remaining solid from the rig and dissolved it in aqueous solution (pH 7.6). The chromatogram of the aqueous solution with the remaining Ilomastat compound on day 30 and the chromatogram of the aqueous solution collected from the rig at the first time point were compared. Both chromatograms were very similar suggesting that no decomposition had occurred during the 30 day period (data not shown).

### Example 1

Ability of Irradiated Ilomastat Powder and of Irradiated Ilomastat Tablet Directly Dissolved in Media without DMSO to Inhibit Contraction in vitro.

The gels of all the three treatment categories (normal media, non-irradiated and irradiated Ilomastat) did not start contracting immediately. For that reason no significant changes were shown in the three treatment groups up to day 1. From day 2, the gels started to contract and the inhibitory effect of both the irradiated and non-irradiated Ilomastat was apparent. There was a statistically significant difference in contraction between the negative control group and the Ilomastat group up to day 7 that the experiment was terminated.

TABLE 5
Contraction (%) of HTF collagen I gels over time

| | DAY 0 | DAY 1 | DAY 2 | DAY 3 | DAY 4 | DAY 5 | DAY 6 | DAY 7 |
|---|---|---|---|---|---|---|---|---|
| NORMAL MEDIA | 0 | 12.23 | 52.16 | 66.52 | 69.47 | 69.1 | 69.93 | 72.03 |
| NON IRRADIATED | 0 | 11.96 | 27.19 | 32.97 | 39.9 | 41.66 | 42.03 | 43.19 |
| IRRADIATED ILOMASTAT | 0 | 12.51 | 23.65 | 28.44 | 30.8 | 32.23 | 33.76 | 36.12 |

2^{nd} in vitro Experiment

TABLE 6
Contraction (%) of HTF collagen I gels over time

| | Day 1 | | | | | |
|---|---|---|---|---|---|---|
| | Group | Group | Group | Group | | standard |
| | 1 | 2 | 3 | 4 | Average | error |
| Normal Media | 39.06 | 42.25 | 47.43 | 28.54 | 39.32 | 3.565058122 |
| Non-Irradiated Ilomastat | 13.03 | 19.45 | 20.69 | 24.23 | 19.35 | 2.09071577 |
| with DMSO | | | | | | |
| Irradiated Ilomastat tablet | 23.24 | 24.98 | 21.44 | 25.46 | 23.78 | 0.817728962 |
| | Day 2 | | | | | |
| | Group | Group | Group | Group | | standard |
| | 1 | 2 | 3 | 4 | Average | error |
| Normal Media | 55.56 | 58.93 | 61.1 | 54.12 | 57.4275 | 1.418302026 |
| Non-Irradiated Ilomastat | 19.75 | 22.77 | 21.37 | 26.63 | 22.63 | 1.314107426 |
| with DMSO | | | | | | |
| Irradiated Ilomastat tablet | 28.57 | 26.24 | 23.93 | 26.99 | 26.4325 | 0.863325151 |
| | Day 3 | | | | | |
| | Group | Group | Group | Group | | standard |
| | 1 | 2 | 3 | 4 | Average | error |
| Normal Media | 69.95 | 74.05 | 72.06 | 59.3 | 68.84 | 2.941247917 |
| Non-Irradiated Ilomastat | 20.44 | 24.14 | 21.55 | 27.17 | 23.325 | 1.339744676 |
| with DMSO | | | | | | |
| Irradiated Ilomastat tablet | 30.97 | 27.16 | 24.66 | 27.39 | 27.545 | 1.161244442 |
| | Day 4 | | | | | |
| | Group | Group | Group | Group | | standard |
| | 1 | 2 | 3 | 4 | Average | error |
| Normal Media | 82.26 | 81.72 | 82.54 | 69.95 | 79.1175 | 2.73753794 |
| Non-Irradiated Ilomastat | 21.15 | 25.19 | 22.11 | 28.11 | 24.14 | 1.412473469 |
| with DMSO | | | | | | |
| Irradiated Ilomastat tablet | 31.39 | 30.49 | 25.87 | 28.32 | 29.0175 | 1.101253257 |
| | Day 5 | | | | | |
| | Group | Group | Group | Group | | standard |
| | 1 | 2 | 3 | 4 | Average | error |
| Normal Media | 84.8 | 84.45 | 85.82 | 72.12 | 81.7975 | 2.897043657 |
| Non-Irradiated Ilomastat | 23.36 | 25.33 | 22.96 | 29.85 | 25.375 | 1.412371982 |
| with DMSO | | | | | | |
| Irradiated Ilomastat tablet | 32.02 | 31.86 | 25.99 | 32.47 | 30.585 | 1.374865422 |
| | Day 6 | | | | | |
| | Group | Group | Group | Group | | standard |
| | 1 | 2 | 3 | 4 | Average | error |
| Normal Media | 85.21 | 85.89 | 86.65 | 78.45 | 84.05 | 1.690242695 |
| Non-Irradiated Ilomastat | 24.42 | 27.9 | 24.9 | 33.35 | 27.6425 | 1.835769566 |
| with DMSO | | | | | | |
| Irradiated Ilomastat tablet | 34 | 32.03 | 27.76 | 34.39 | 32.045 | 1.358533111 |
| | Day 7 | | | | | |
| | Group | Group | Group | Group | | standard |
| | 1 | 2 | 3 | 4 | Average | error |
| Normal Media | 85.91 | 86.9 | 86.93 | 80.47 | 85.0525 | 1.382621144 |
| Non-Irradiated Ilomastat | 25.07 | 29 | 24.9 | 34.04 | 28.2525 | 1.922235588 |
| with DMSO | | | | | | |
| Irradiated Ilomastat tablet | 35.36 | 32.99 | 27.92 | 36.63 | 33.225 | 1.719558018 |

### Example 2

The Effectiveness of Ilomastat Tablet in the in vivo Experiment

1. Clinical Observations

The bleb in the rabbit that received the ethylcellulose tablet (control) failed on day 10 after glaucoma filtration surgery. In contrast, the blebs of the three rabbits that received the Ilomastat tablet did not fail. After 30 days the experiment as planned was terminated. In one rabbit the scleral sutures broke on day 7 and the tube fell into the anterior chamber. When this occurs the normal expectation is that the bleb will fail; however, a well structured bleb surprisingly remained present in this rabbit until day 30. No corneal epitheliopathy was observed in the rabbits of the treated and control groups. Additionally, the conjunctiva over the bleb area was normal and not avascular. Avascular blebs have been observed after the use of MMC in glaucoma filtration surgery. Moreover, no soft eyes were observed.

2. Detection of Ilomastat in the Fluid Samples Collected from Rabbits on Day 30

Using the HPLC method described above, no Ilomastat was detected in the aqueous humor from the anterior chamber, vitreous or blood samples collected from the left (operated) eye of the rabbits on day 30. The retention time of Ilomastat as it was previously mentioned is 6.5-8 minutes and around that time point no peak was detected. These observations indicate that for GFS treatment outflow of the Ilomastat occurred with the result that any potential local toxicity can be avoided.

Conclusions

The inventors observed a prolonged release of Ilomastat from the tablets tested. These tablets were fabricated without use of any excipients. During the release period (30 days), a therapeutic dose of Ilomastat (10 μM) was achieved. The use of a solid form of Ilomastat provides a method of preventing tissue scarring that does not require multiple injections. In contrast to previous in vitro and in vivo experiments, the inventors avoided using DMSO throughout the experiments, as it has not been approved for ocular clinical use.

A very important issue is the need to sterilize the tablet. The effects of irradiation in other metalloproteinase inhibitors, such as Captopril, have been evaluated (Engalytcheff et al. 2004; Engalytcheff, Vanhelleputte, & Tilquin 2004). Degradation of Captopril caused from irradiation was not significant. The inventors have found that the degradation of Ilomastat caused by the 25 KGys gamma radiation dose was not significant and is within the acceptable limits as defined by the European and US Pharmacopoeias. Gamma irradiation provides a significant advantage to perform Ilomastat tablet sterilization in their package, as the package can be opened in the operating room without any further process needed to take place between gamma irradiation and the placement of the tablet in the subconjunctival space.

Furthermore, the inventors tested the effectiveness of irradiated Ilomastat to inhibit collagen I gel contraction and observed significant inhibition compared to the negative control and inhibition at about the same levels as the positive control. Although irradiated Ilomastat seems to be slightly more potent in inhibiting gel contraction than the non-irradiated Ilomastat, this difference is not statistically significant. The inventors believe that the main reason for this difference could be the use of slightly higher number of cells in the non-irradiated Ilomastat gels. The number of cells used for each gel is unfortunately a parameter that is not very accurate and this can result in slight differences in contractions being observed.

Finally, in the in vivo GFS model, the inventors observed that Ilomastat inhibited scarring after GFS in all the rabbits until day 30 when the experiment was required to be terminated. Another encouraging result was the lack of detection of Ilomastat in the aqueous humor, vitreous and blood. Thus Ilomastat would be expected not to interfere with other eye structures and other parts of the body.

The use of Ilomastat and other MMP inhibitors in a solid tablet form for implantation at the site of surgery has been shown to have significant beneficial advantages for reducing and preventing tissue scarring.

### Example 3

In vitro Experiment Using 5-FU

As indicated above, a tablet of solid 5-FU was fabricated using the same technique as described above. The dissolution rate of the tablet was then determined using the same rig as described above.

Results

Calibration Curve

A calibration curve for 5-FU dissolution at 7.6 pH aqueous solution without DMSO is shown in FIG. 6. The curve was generated by measurement of the 5-FU peak in the HPLC reader using the software PC Chrom+.

The calibration curve for 5-FU was created in the same manner as that for Ilomastat.

Release Profile

The first tablet (tablet A) had a diameter of 3 mm, thickness of 0.71 mm and a weight of 7.1 mg. The second tablet (tablet B) had the same diameter, thickness of 0.88 mm and weight of 8.7 mg. The third tablet (tablet C) had diameter of 3 mm, thickness of 0.76 mm and weight of 7 mg.

Each tablet was placed into the rig as described above and liquid samples analysed by HPLC and concentration of 5-FU was determined using the calibration curve.

The data from tablets A, B and C was averaged and the release profiles shown graphically in FIGS. 7 and 8.

The data shows a prolonged release of 5-FU. These tablets were fabricated without use of any excipients. During the release period (25 hours), a substantially constant therapeutic dose of 5-FU was achieved. The use of a solid form of 5-FU provides a prolonged release that is of benefit in preventing tissue scarring.

### Example 4

Sustained Release of Active Agent from Excipient-Free Tablets

FIGS. 9 to 13 show the results obtained with a variety of chemically unrelated active agents, formulated as excipient-free tablets (as described above), using the flow-through dissolution rig. In each case, (a) shows the cumulative release of drug as a percentage of total drug content in the tablet, whereas (b) shows the concentration in the flow-through cell at each point in time.

It will be observed that each of the tablets tested produces essentially zero order (i.e. constant rate) release of drug. This is illustrated by the linear traces in (a) and the (for the most part) essentially flat traces in (b). This confirms that such tablets would be capable of producing essentially constant, therapeutically relevant levels of drug in an implantation site in vivo, over a period of many days. Even the dosage form containing the significantly more soluble drug 5-FU (FIG. 9) is shown to produce essentially linear release of drug over a period of many hours. These results show that, compared to conventional dosage forms for local administration of drugs to the eye (e.g. eye drops or ocular injectables), the residence time of the dosage forms of the invention would be much greater. This would provide significant clinical advantage since the active agent would be present in the tissue for far longer.

### Example 5

Tablet Composition Containing Solid Antibody

The aqueous injectable formulation of bevacizumab (marketed as Avastin) was used as starting material. To remove excipients (e.g. trehalose), pharmaceutical Avastin (50 μl of 25 mg/ml) was added to a spin column with membrane of cutoff of 10000 daltons (Vivaspin 10000 from Vivascience). Distilled water (4 ml) was added and the column centrifuged for 4 minutes at 4000 rpm. This step was repeated twice. Removal of trehalose was confirmed by thin layer chromatography (TLC; aqueous methanol 90%). Different concentrations of trehalose and intact Avastin were used as control. TLC film was dipped into a mixture of sulfuric acid (10%) and ethanol (90%) and then heated.

The obtained solution of bevacizumab was then freeze dried to isolate the antibody as a powder which was then used to fabricate a 1.25 mg bevacizumab tablet (as described above, and containing essentially only the freeze dried antibody). A release profile is shown in FIG. 14 where total protein (BCA assay—upper line in FIG. 14) and protein that binds to a VEGF chip (determined using a Biacore biosensor) are compared. These data confirms that the antibody is released from the tablet, potentially over a period of days, and also confirms that a significant portion of the antibody retains its VEGF-binding activity. The data for ‘active protein’ release is re-plotted in FIG. 15 with actual data points shown.

FIG. 16 shows the size exclusion chromatography (SEC) results for bevacizumab reconstituted from an excipient-free tablet according to the invention (labeled b), compared to untreated Avastin solution (labeled a), and compared to bevacizumab reconstituted from a tablet according to the invention, but with excipients not removed (unlabeled trace). In brief, the SEC conditions were as follows:

Sample injection volume: 150 μl

Mobile phase: phosphate buffer (NaH₂PO₄, 25 mM, pH 6.8 and NaCl 150 mM)

Flow rate: 1 mL/min

Column: (Hiload™, Superdex™ 200)

UV detector: 280 nm

The data of FIG. 16 confirm that the molecular weight of the tableted bevacizumab is not changed compared to the Avastin control solution, i.e. the purification and tableting steps do not lead to aggregation of the antibody. As with the results shown in Example 4, the implantation site residence time of a dosage form prepared according to the present example would be significantly greater than that of, e.g. eye drops or ocular injectables. This would provide significant clinical advantage.

Manipulation of the release profile of antibody-containing compositions of the invention, such as tablets, may be achieved by the incorporation of certain excipients. This approach may also lead to improvements in the retention of antibody activity. FIG. 17 shows the effect of adding 1.75 mg hyaluronic acid (Healon) per tablet. The concentrations achieved in the first 48 hours or so of release are markedly higher than from an equivalent excipient-free tablet (see FIGS. 14 and 15). This effect could be due to increased release per se of antibody, and/or could be related to an improved retention of antibody binding in the hyaluronic acid-containing tablet. Note that the biphasic release profile shown in FIG. 17 is believed to be an artifact of the dissolution rig employed.

When the amount of hyaluronic acid is increased to 3.5 mg per tablet, the antibody release is dramatically reduced. Again, artifacts of the dissolution apparatus could be reflected in this data (small beads of the formulation were observed to stick to the sides of the flow cell), but it is believed that the higher hyaluronic acid content leads to a more sustained and steady release of the antibody. The dissolution profile of the antibody tablets can thus be tailored by an appropriate choice of excipients.

All documents cited herein are incorporated herein by reference.

### REFERENCES

1. Am. J. Ophthalmol., vol. 1996 April; 121, no. 4, pp. 349-366.

2. The United States Pharmacopoeia USP 24. 2000a. United States Pharmacopeial Convention, Rockville, Md., 2000.

3. Pharmacope'e Europe'enne 4e 'me Edition, pp. 439-444. 2000b. Strasbourg.

4. Cordeiro, et al., 2000, Curr. Opin. Ophthalmol, vol. 11, no. 2, pp. 121-126.

5. Dahlmann et al., 2005, Opthalmol Clin North Am., vol. 18, pp. 539-59.

6. Daniels, et al., 2003, Invest Ophthalmol Vis. Sci., vol. 44, no. 3, pp. 1104-1110.

7. Daniels, et al., 1998, Microsc. Res. Tech., vol. 42, no. 5, pp. 317-333.

8. Doyle, et al., 1993, Invest Ophthalmol Vis. Sci., vol. 34, no. 12, pp. 3313-3319.

9. Engalytcheff, et al., 2004, Radiat. Res., vol. 162, no. 6, pp. 616-622.

10. Engalytcheff, et al., 2004, Pharm. Res., vol. 21, no. 7, pp. 1103-1108.

11. Galardy, et al., 1994a, Ann N.Y. Acad. Sci., vol. 732, pp. 315-323.

12. Galardy, et al., 1994c, Ann N.Y. Acad. Sci., vol. 732, pp. 315-323.

13. Galardy, et al., 1994b, Ann N.Y. Acad. Sci., vol. 732, pp. 315-323.

14. Galardy, et al., 1994d, Cancer Res., vol. 54, no. 17, pp. 4715-4718.

15. Galardy, et al., 1994e, Cancer Res., vol. 54, no. 17, pp. 4715-4718.

16. Glasspool, et al., 2001b, Breast, vol. 10, no. 5, pp. 368-378.

17. Glasspool, et al., 2001a, Breast, vol. 10, no. 5, pp. 368-378.

18. Harris, et al., 1981, Nature, vol. 290, no. 5803, pp. 249-251.

19. Kawashima, et al., 1998, Curr. Eye Res., vol. 17, no. 4, pp. 445-451.

20. Khaw, et al., 1994, Eye, vol. 1994; 8, no. Pt 2, pp. 188-195.

21. Khaw, et al., 1992, Invest Ophthalmol Vis. Sci., vol. 33, no. 6, pp. 2043-2052.

22. Mietz, et al., 2003, Invest Ophthalmol Vis. Sci., vol. 44, no. 12, pp. 5182-5188.

23. Porter, et al., 1998, Wound. Repair Regen., vol. 6, no. 2, pp. 157-166.

24. Scott, et al., 1998, FEBS Lett., vol. 441, no. 1, pp. 137-140.

25. Skiles, et al., 2001, Curr. Med. Chem., vol. 8, no. 4, pp. 425-474.

26. Skuta, et al., 1992, Ophthalmology, vol. 99, no. 3, pp. 438-444.

27. Wong, et al., 2003, Invest Ophthalmol Vis. Sci., vol. 44, no. 3, pp. 1097-1103.

28. Wong, et al., 2005, Invest Ophthalmol Vis. Sci., vol. 46, no. 6, pp. 2018-2022.

29. Wong, et al., 2002, Surv. Ophthalmol, vol. 47, no. 3, pp. 239-256.

1-58. (canceled)

59. A solid, implantable dosage form comprising a therapeutically active agent in solid form and one or more pharmaceutically acceptable excipients, wherein the one or more excipients do not lead to a significant delay or prolongation of the release of active agent as compared to an equivalent dosage form containing no excipients.

60. A dosage form according to claim 59, which is sterilized.

61. A dosage form according to claim 59, wherein the one or more excipients are biodegradable and/or bioresorbable following implantation.

62. A dosage form according to claim 59, wherein the one or more excipients do not control the release of the active agent by means of the chemical or biochemical degradation of one or more of the excipients.

63. A dosage form according to claim 59, which is prepared by compression.

64. A dosage form according to claim 59, having a volume of between 0.1 mm³ and 1.5 cm³, and/or having a maximum dimension of 5 mm or less, and/or having a weight of 10 mg or less.

65. A dosage form according to claim 59, wherein the active agent is substantially water insoluble.

66. A dosage form according to claim 59, which is substantially free of excipients.

67. A solid, implantable dosage form comprising a therapeutically active agent in solid form, wherein the active agent is selected from the group consisting of a matrix metalloproteinase inhibitor, an anticancer agent, a steroid, an antibiotic, an antibody molecule, an anti-inflammatory agent, and an anti-scarring agent.

68. A dosage form according to claim 67, further comprising one or more pharmaceutically acceptable excipients.

69. A dosage form according to claim 68, wherein the one or more excipients are biodegradable and/or bioresorbable following implantation.

70. A dosage form according to claim 68, wherein the one or more excipients do not control the release of the active agent by means of the chemical or biochemical degradation of one or more of the excipients.

71. A dosage form according to claim 67, wherein the active agent is a matrix metalloproteinase inhibitor.

72. A dosage form according to claim 71, further comprising one or more pharmaceutically acceptable excipients.

73. A dosage form according to claim 71, wherein the matrix metalloproteinase inhibitor is a hydroxamic acid derivative that binds reversibly to zinc in the active site of matrix metalloproteinases.

74. A dosage form according to claim 71, wherein the matrix metalloproteinase inhibitor is a right side binder.

75. A dosage form according to claim 71, wherein the matrix metalloproteinase inhibitor is selected from the group consisting of ilomastat, batimastat, marimastat, prinomastat, tanomastat, Trocade (cipemastat), AG 3340, CGs227023A, BAY 12-9566, and BMS-275291, or any functional derivatives thereof

76. A dosage form according to claim 67, wherein the anticancer agent is 5-fluorouracil, the steroid is selected from triamcinolone and dexamethasone, and the anti-inflammatory agent is naproxen.

77. A dosage form according to claim 67, wherein the active agent is substantially water insoluble.

78. A dosage form according to claim 67, which is sterilized.

79. A dosage form according to claim 67, which is prepared by compression.

80. A dosage form according to claim 67, having a volume of between 0.1 mm³ and 1.5 cm³, and/or having a maximum dimension of 5 mm or less, and/or having a weight of 10 mg or less.

81. A dosage form according to claim 67, further comprising one or more additional therapeutically active agents, wherein the one or more additional agents are not in solid form.

82. A dosage form according to claim 67, wherein the dosage form is an antibody molecule.

83. A dosage form according to claim 82, wherein the antibody molecule is a monoclonal antibody.

84. A dosage form according to claim 82, wherein the antibody molecule is an antibody indicated for the treatment of a neoplastic disease.

85. A dosage form according to claim 84, wherein the antibody molecule is an anti-VEGF antibody.

86. A method of locally treating a disease or condition in a patient in need thereof, the method comprising administering a solid dosage form according to claim 67 to said patient, by implantation, in an amount sufficient to treat the disease or condition.

87. A method according to claim 86, wherein the condition is scarring.

88. A method according to claim 87, wherein the dosage form is administered by ocular, periocular or intraocular implantation.

89. A method according to claim 88, wherein the dosage form is implanted in the subconjunctival space.

90. A method according to claim 87, wherein the scarring is that following glaucoma filtration surgery.

91. A method according to claim 86, wherein the active agent is substantially water insoluble.

92. A method according to claim 86, wherein the active agent is a matrix metalloproteinase inhibitor.

93. A method according to claim 92, wherein the matrix metalloproteinase inhibitor is formulated as a solid, implantable medicament for local implantation.

94. A method according to claim 86, wherein the solid dosage form further comprises one or more pharmaceutically acceptable excipients.

95. A method according to claim 86, wherein the implantation is local implantation.

96. A method according to claim 86, wherein the active agent is sterilized.

97. A method according to claim 86, wherein the disease is a neoplastic disease.

98. A method of manufacturing a dosage form according to claim 71, the method comprising:
i. forming a compressed dosage form containing the matrix metalloproteinase inhibitor, and
ii. sterilizing the compressed dosage form by irradiating it with gamma radiation.

99. A method according to claim 98, wherein the compressed dosage form further comprises one or more pharmaceutically acceptable excipients.

100. A kit comprising a dosage form according to claim 71 and surgical equipment necessary for performing glaucoma filtration surgery.
